(19) United States
(12) Patent Application Publication
(10) Pub. No.: US 2008/0149479 A1
(43) Pub. Date: Jun. 26, 2008

(54) Sensor for Detection of Single Molecules

(75) Inventors: Linda Olofsson, Onsala (SE); Niklas Hansson, Askim (SE); Niklas Olofsson, Goteborg (SE); Anders Lundgren, Askim (SE); Patrik Nordberg, Goteborg (SE)

Correspondence Address: YOUNG & THOMPSON, 209 Madison Street, Suite 500, ALEXANDRIA, VA, 22314, US

(73) Assignee: MIDORION AB, Goteborg (SE)

(21) Appl. No.: 11/884,025

(22) PCT Filed: Feb. 20, 2006
(86) PCT No.: PCT/SE06/00227

§ 371 Date: Oct. 11, 2007

Related U.S. Application Data
(60) Provisional application No. 60/656,831, filed on Feb. 28, 2005.
(60) Provisional application No. 60/657,590, filed on Mar. 2, 2005.

(30) Foreign Application Priority Data
Feb. 18, 2005 (SE) 0500378-5
Feb. 18, 2005 (SE) 0500379-3

(51) Int. Cl. G01N 27/26; C07F 1/12; B05D 5/12; C12M 1/40

(52) U.S. Cl. 204/403.14; 556/116; 204/400; 204/403.01; 427/77

(57) ABSTRACT

A single electron transistor device for sensing at least one particle, includes at least two electrodes positioned with a gap formed between the electrodes and an activation object positioned in the gap with an insulating layer between the activation object and each electrode. The activation object which is able to transfer electrons is arranged with at least one binding structure bonded to it for receiving the at least one particle. The electrodes are formed with an inter distance of less than 50 nm and the electrodes are connectable directly or indirectly to a signal acquisition system. The sensing device is arranged to allow a tunnelling current proportional to the presence of the at least one particle in the binding structure, to flow through the activation object. A method, and system using a single electron transistor device fabricated with micro/nano fabrication methods are also disclosed.

### TECHNICAL FIELD

The present invention relates to a sensor for sensing of low concentrations or single units of particles and in particular to a device, method, and system using a single electron transistor (SET) device fabricated with micro/nano fabrication methods.

### BACKGROUND OF THE INVENTION

Sensor technologies have been widely studied. Since the advent of MEMS technology, the research and development area has been focused in finding different solutions for sensing different parameters and characteristics using small scale electronic devices fabricated in MEMS technology. The sensors have often been adapted to measure physical characteristics such as acceleration for gyroscopic sensors. However, there have been only few attempts on finding sensors that have been adapted to measure the presence of single or low concentrations of molecules or particles. These have been experimental systems and which have been found in special research facilities and generally not available as commercial instrumentation.

Patent application publication WO 02/42757 describes an extremely sensitive transducer, a single electron transistor (SET) that may be used for highly sensitive biosensing. In contrast to the ordinary transistors of today (MOSFETs), where the description does not require quantum mechanics, the single-electron transistors are based on a quantum phenomenon, namely the tunneling effect. The tunneling effect is observed when particles, in this case, electrons impinge on a potential barrier. Classically there is no chance that the particle will pass the barrier if the energy of the particle is less than the energy of the barrier. But in the quantum world there is still a probability that the particle will pass the barrier. The probability of tunneling decreases exponentially with the height and width of the barrier. The number of electrons impinging the barrier every second is huge, but if the probability of transmission is very low only a few of the electrons will pass the barrier. In a structure that consists of two electrodes separated from a small island by two thin barriers, electrons can tunnel one by one. This is the foundation of a single-electron tunneling transistor (SET). The first real fabrication of a SET was reported in 1987 (Fulton T. A., Dolan G. J. Observation of single-electron charging effects in small tunnel junctions. Phys. Rev. Lett. (1987), 59, p 109).

The advantages of using a SET for biosensing has been confirmed theoretically in a paper from 2004 (The dnaSET: A Novel Device for Single Molecule DNA Sequencing. IEEE transaction on electron devices, 51, 12). In order to use a SET as transducer for biosensing it needs to be operated at room temperature in a liquid environment. The SET also needs to be chemically modified and biologically functionalized in order to perform biosensing. Similar structures have been fabricated but not in the context of biosensing. Fabrication of one such structure is reported by Bezryadin et al. (A. Bezryadin, C. Dekker and G. Schmid Electrostatic trapping of single conducting nanoparticles between nanoelectrodes, Appl. Phys. Lett. (1997), 71(9):p. 1273.). Fabrication of another similar structure is reported by Klein et al. (D. Klein, R. Roth, A. Lim, A. Alivisatos and P. McEuen A single-electron transistor made from a cadmium selenide nanocrystal, Nature (1997), 389:p. 699). Yet another similar structure is fabricated and reported by Olofsson (L. Olofsson Nanofabrication of single electron transistors and evaluation of miniature biosensors, PhD thesis, Chalmers University of Technology (2003).). None of these structures deals with the problems of adapting the SET for the biosensing application. The current invention deals with this problem of adapting and optimizing the SET for biosensing.

WO 02/42757 uses primarily a gated system wherein the electron flow between the electrodes is controlled by a separate gate voltage. This requires an additional complexity of manufacture and may be difficult to implement for a plurality of electrodes. The solution described in WO 02/42757 is not optimized for bio sensing of molecules in a solution.

It is therefore an object of the present invention to remedy at least some of these problems,

### SUMMARY OF THE INVENTION

The object of the present invention is to provide a device, system, and methods for fabrication of such a device that is optimized for bio sensing and capable of receiving samples in a solution flowed over the sensing part. This is provided in several aspects of the present invention.

The present invention is a high-throughput device for biosensing. The device can be used effectively and quantitatively to determine and study interaction between molecules, for instance biomolecules.

The device involves at least one SET that has been adapted into a transducer that is optimized for biosensing.

One aspect of the invention involves covering electrodes with linking molecules that reduce leakage currents and bind specifically to the activation object.

Another aspect of the invention involves the activation object is specifically chosen for biosensing. It is functionalized and fabricated in aqueous solution.

Another aspect of the invention involves covering of all but the active parts of the electrodes with an insulating layer. This coverage will increase the sensitivity of the biosensor and also reduce distortion during measurements.

Another aspect of the invention involves the fabrication method of the device.

This will now be summarized in more detail in the following aspects, wherein a first aspect of the present invention. an electronic sensing device for sensing at least one particle is provided, comprising at least two electrodes positioned with a gap formed between the electrodes and an activation object positioned in the gap with an insulating layer between the activation object and each electrode; the activation object being able to transfer electrons and arranged with at least one binding structure bonded to the activation object for receiving the at least one particle characterized in that the electrodes are formed with an inter distance of less than 50 nm and the electrodes being connectable directly or indirectly to a signal acquisition system; the sensing device is arranged to allow a tunnelling current proportional to the presence of the at least one particle in the binding structure, to flow through the activation object.

The device may further comprise an insulating layer formed on at least part of at least one electrode on a surface of the electrode facing particles to be sensed.

The insulating layer may be formed in part by angle evaporation on a double resist mask.

The insulating layer may be made of SiO₂, titanium oxide, aluminium oxide, chromium oxide, iron oxide, beryllium oxide, ceramics, polystyrene or teflon.

The device may further comprise a sticking layer formed under at least part of each electrode. The sticking layer may be made of at least one of chromium, titanium, NiCr, or aluminium oxide. The activation object may be a nano sized particle made of a metal or a conducting compound.

The device activation object may be made of at least one of gold, titanium, aluminium, copper, iron, silver, palladium, cobalt or cadmium selenide.

The activation object may be stabilized by a stabilizing agent. The stabilizing agent may be citrate.

The activation object may be stabilized and/or functionalized with a self-assembling monolayer (SAM). The self-assembled monolayer, SAM may comprise one or more thiols. The self-assembled monolayer, SAM may comprise one or more alkanethiols. The self-assembled monolayer, SAM may be assembled from hydrophilic substituted alkanethiols or hydrophobic alkanethiols.

The stabilized activation object may have a diameter of less than 20 nm, more preferably a diameter of less than 18 nm, more preferably a diameter of less than 16 nm, more preferably a diameter of less than 14 nm, more preferably a diameter of less than 12 nm, more preferably a diameter of less than 10 nm, more preferably a diameter of less than 8 nm, more preferably a diameter of less than 6 nm, and most preferably a diameter of less than 4 nm.

The activation object may be functionalized by binding a binding structure.

The stabilized activation object in may be functionalized by exchange mediated functionalisation.

The binding structure may be a compound from the group comprising water solvable ionic or zwitterionic compounds.

The binding structure may be a molecular structure having functional groups chosen from the group comprising thiol, sulphide, amine, carboxylate, cyanide, diphenylphosphine and/or pyridine functional groups.

The binding structure may be chosen from the group comprising ions, atoms, molecules, low-molecular compounds, nucleotides, DNA-fragments, DNA-sequences, amino acids, peptides, proteins, antibodies, enzymes, receptors, and/or molecular imprinted polymers.

The activation object may be functionalized with Avidin. The avidin functionalized activation object may be bound to a biotinylated protein or protein fragment. The activation object may been functionalized with cysteine or cystine.

The surfaces of the electrodes have been functionalized.

The functionalized electrodes may be covered with a self-assembled monolayer, SAM.

The self-assembled monolayer, SAM may comprise one or more alkanethiols with 16 or less carbon atoms, preferably alkanethiols with 14 or less carbon atoms, preferably alkanethiols with 12 or less carbon atoms, preferably alkanethiols with 10 or less carbon atoms, preferably alkanethiols with 8 or less carbon atoms, preferably alkanethiols with 6 or less carbon atoms, preferably alkanethiols with 4 or less carbon atoms. The alkanethiol may be a substituted alkanethiol, and wherein the alkanethiol may be a carboxylate terminated alkanethiol, a mercaptohexadecanoic acid, or a mercaptopropionic acid.

The activation object may be a functionalized activation object as claimed in one or more of claims 16-24 immobilized to an electrode functionalized as claimed in one or more of claims 25-31.

The functionalized activation object may be immobilized to a functionalized electrode by covalent immobilization or by carbodiimide coupling.

The functionalized activation object may be immobilized to a functionalized electrode by glutaraldehyde coupling.

The functionalized activation object may be covalently coupled to a binding structure.

The binding structure may be one of the group comprising nucleotides, DNA-fragments, DNA-sequences, amino acids, peptides, proteins, antibodies, enzymes, receptors, molecular imprinted polymers.

The binding structure may be covalently coupled to a through the reactive groups of amino acid chosen from the groups comprising lysine, the N-terminal of the peptide with primary amines, aspartate, glutamate, the C-terminal with carboxylate groups and/or cysteine

The binding structure may be covalently coupled by carbodiimide coupling.

The binding structure may be covalently coupled by glutaraldehyde coupling.

Another aspect of the present invention, a method for producing a cystine functionalized activation object (4) is provided, characterized in that;

a) a solution of citrate stabilized gold nanoparticles having a mean diameter of less than 20 nm is mixed with equal volumes of a saturated cystine solution
b) incubating the mixture in room temperature for 8-12 hrs
c) centrifuging the mixture forming a pellet
d) redissolving the pellet in water.

Yet another aspect of the present invention, a cystine functionalized activation object (4) is provided, prepared by

a) mixing equal volumes of citrate stabilized gold nanoparticles having a mean diameter of less than 20 nm with equal a saturated cystine solution
b) incubating the mixture in room temperature for 8-12 hrs
c) centrifuging the mixture forming a pellet
d) redissolving the pellet in water.

Still another aspect of the present invention, a use of the cystine functionalized particle in claim 42 as the activation object (4) in the device of claim 1 is provided.

Yet another aspect of the present invention, a system for measuring low quantities of molecules or particles is provided, comprising:
an electronic sensing device for sensing particles, comprising at least two electrodes positioned with a gap formed between the electrodes and an activation object positioned in the gap with an insulating layer between the activation object and each electrode; the activation object being able to transfer electrons and arranged with at least one binding structure bonded to the activation object for receiving at least one particle characterized in that the electrodes are formed with an inter distance of less than 50 nm and the electrodes being connectable directly or indirectly to a signal acquisition system; the sensing device is arranged to allow a tunnelling current proportional to the presence of particle or particles in the binding structure, to flow through the activation object;
electronics for signal processing; and
a processing device for control of measurement and signal acquisition for processing and analysis of measured signals.

The system further comprising a holder for holding the electronic sensing device and arranged with a quick release lock. The system further comprising a delivery system for providing particles to be measured to the electronic sensing device.

Yet another aspect of the present invention, a method of fabricating a gap between electrodes in an electronic sensing device for sensing particles is provided, comprising the steps of:
forming a first electrode onto a surface;
forming an aluminium layer on the first electrode;
oxidizing the aluminium layer;
forming a second electrode at least partly over the first electrode and the oxidized aluminium layer;
removing a part of the second electrode located on the oxidized aluminium layer; and
removing the oxidized aluminium layer and the aluminium layer from the first electrode.

Another aspect of the present invention, an electronic sensing device for sensing particles is provided, comprising at least two electrodes positioned with a gap formed between the electrodes and a tunnelling object positioned at least partly in the gap with an insulating layer between the tunnelling object and each electrode; the tunnelling object being able to transfer electrons, the device further comprising a gate arranged to receive particles to be sensed, characterized in that the electrodes are formed with an inter distance of less than 50 nm and the electrodes being connectable directly or indirectly to a signal acquisition system; the sensing device is arranged to allow a tunnelling current proportional to the presence of particle or particles on the gate, to flow through the tunnelling object.

Another aspect of the present invention, a method of fabrication of nanogaps according to a process wherein a double resist layer is used is provided, comprising the steps of:
patterning a top resist with electrons and developing;
developing the non-electron sensitive bottom resist layer under the top resist and forming a thin bridge of the top resist;
defining, during evaporation the distance between two evaporated electrodes, the width of the resist bridge;
forming, due to migration, grains between the electrodes; and
forming a nanogap since the grains extend the electrodes and the nanogap is formed between grains.

The grains may be modified by a plasma.

Yet another aspect of the present invention, a method of fabricating nanogaps is provided, comprising the steps of:
evaporating a first electrode onto a surface;
forming an oxidized aluminium layer on said first electrode;
forming a second electrode on said surface and partly on said oxidized aluminium layer; and
removing said oxidized aluminium layer forming a gap between said first and second electrodes.

### BRIEF DESCRIPTION OF THE DRAWINGS

In the following the invention will be described in a non-limiting way and in more detail with reference to exemplary embodiments illustrated in the enclosed drawings, in which:

FIG. 1a illustrates schematically a sensor device according to the present invention;

FIG. 1b illustrates schematically a close up in a side view of a sensing part of FIG. 1a;

FIG. 2 illustrates schematically a sensing system according to the present invention;

FIG. 3 illustrates schematically a processing device according to the present invention;

FIG. 4 illustrates schematically angle evaporation of insulating layer according to the present invention;

FIG. 5 illustrates schematically how electrodes may be positioned according to the present invention.

FIG. 6 illustrates schematically an I-V curve taken using the present invention;

FIG. 7 illustrates schematically a method of fabricating a gap according to the present invention;

FIG. 8 illustrates schematically another method of fabricating a gap according to the present invention;

FIG. 9 illustrates schematically an alternative embodiment of the present invention;

FIG. 10 illustrates in a schematically block diagram a method of fabricating a gap part of the present invention;

FIG. 11 shows the visible spectra obtained for a “raw” 14 nm gold nanoparticle solution;

FIG. 12 shows the visible spectra for larger (14 nm) and smaller (5 nm) AuNPs after sequential ultra centrifugation;

FIG. 13 shows the visible spectra for large (14 nm) AuNPs before and after adsorption of Avidin to the particle surfaces, and after biotin-BSA addition;

FIG. 14 shows the Biacore response for the injection of a diluted solution of Avidin coated AuNPs (14 nm) and a 0.1 mg/ml Avidin solution on a biotin functionalised surface;

FIG. 15 shows the visible spectra for cystine functionalised and non-coated AuNPs (5 nm) before and after the addition of glutaraldehyde to the cuvette;

FIG. 16 shows the different injection steps, for the covalent EDC/NHS mediated immobilisation of cystine modified AuNPs to a carboxylate terminated SAM and the subsequent covalent immobilisation of Avidin to the nanoparticle surface;

FIG. 17 shows the amount of Avidin, and subsequently biotin-BSA, possible to immobilise to the carboxylate SAM and the SAM/AuNP surface; and

FIGS. 18A and B shows SET-biosensing with cystine modified AuNPs.

### DETAILED DESCRIPTION OF PREFERRED EMBODIMENTS

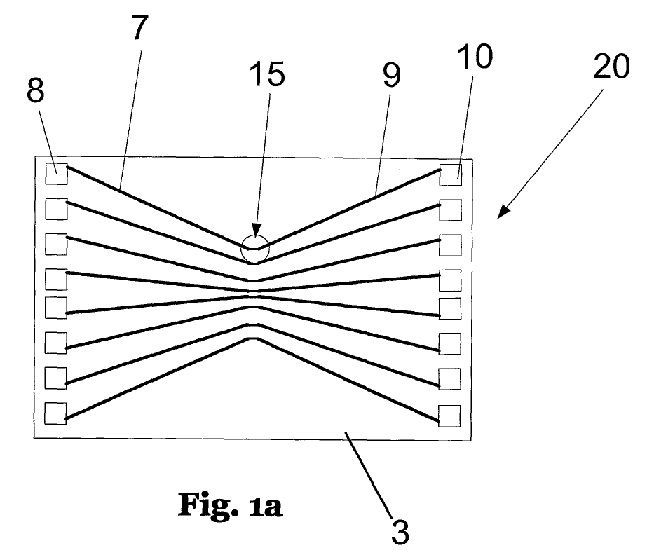

In FIG. 1 reference numeral 20 generally denote a sensing device 20 according to the present invention. The sensing device 20 comprises one or several electrode pairs shown in more detail in FIG. 1b. Each electrode 1, 2, formed on a surface 3 (e.g. a substrate as used in micro/nano lithography fabrication), has a connection conductor 7, 9 connected to a connector 8, 10. Reference numeral 15 shows the area that is shown in more detail in FIG. 1b. A plurality of such electrode pair combinations may be positioned on a chip. Between each electrode 1 and 2 pair a gap 12 is formed wherein an activation object 4 (e.g. a nanoparticle) is positioned or formed through which electrons may flow during measurement. The object 4 may be for instance a metal sphere or sphere like particle, e.g. made of gold. Below an example using gold as receptor island will be used; however, it should be understood by the person skilled in the art that other receptor islands may be used. On the gold particle 4 one or several binding structures 11 may be attached for receiving molecules or particles 13 of interest to detect their presence. On at least part of the electrodes, an insulating layer 6 may be formed in order to decrease the interaction with the environment and thus it is possible to increase the signal to noise ratio. The insulating layer 6 is formed over substantially the entire electrode 1 and subsequent conductor 7, apart from the region close to the gap formed between two electrodes 1, 2. This is to ensure that the receptor island may have sufficient electrical contact with the electrodes (directly or indirectly) allowing for tunnelling current to pass through the receptor island. In order for the electrodes to stick better to the surface a sticking layer 14 may be formed between the electrode and the surface 3. The sticking layer 14 is optional and depends on the configuration of materials used.

The insulating layer 6 over the electrodes 1, 2 has the benefit of reducing interaction with the environment, for instance current leakage to a solution or buffer in contact with the system 20. Further more it has the advantage of an increased sensitivity of the sensor. Since the activation objects does not attach to the insulating layer it is possible to control the number of activation objects. One way to achieve such an insulating layer is by angle evaporation of silicon oxide, silicon dioxide or another insulating material. This layer stops the transport of electrons from the electrodes directly out into the solution (buffer) or sample.

The sensing device 20 may be a disposable unit that can be changed from a sensing system (which will be described later in this document) depending on what sample that is analyzed or if it has degraded in its operation.

### Sensor Operation and Sensing Modes

The operation of the sensing device 20 is as follows: Due to the high sensitivity of a SET and the dimensions of the electrodes and objects, the electrical conductivity will be very sensitive to any molecules or particles in the vicinity of the activation object. 11. In one embodiment a current through the object versus applied voltage curve is measured, a so called IV curve. From the measured curve it is possible to deduce from signal analysis the amount of molecules that is present in the vicinity of the activation object. The IV-curve measurement is the main measurement mode; however, other modes may be used as for instance impedance measurements determining the impedance through the island; however, this technique is not as sensitive as the tunnelling mode, but it may be applicable for measurements of a larger amount of molecules.

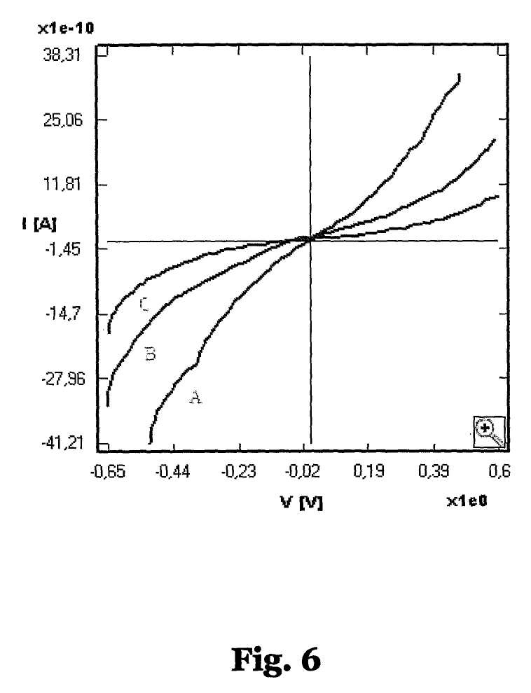

FIG. 6 illustrates a graph containing three IV-curves obtained from measurements: one curve shows a measurement of the device in buffer solution (A), a measurement after addition of Avidin to the system (B) and a measurement after rinsing with buffer and addition of biotinylated albumin to the system (C). Different types of signal analysis can be used on the curves in order to deduce different characterizing parameters, for instance slope detection, zero crossings, Fourier analysis, averaging, and so on.

Molecules or particles to be measured may be present in a solution that is made to flow over the binding structures 11 or they may be present in a gaseous state in contact with the binding structures 11. If a plurality of electrode combinations is formed on a single sensing device 20 each electrode combination 1, 2 may have a receptor island adapted to receive different substances 13, i.e. on the same sensing device 20 different substances may be detected and measured. The binding structure 11 may be for instance a molecular binding structure.

### System Design

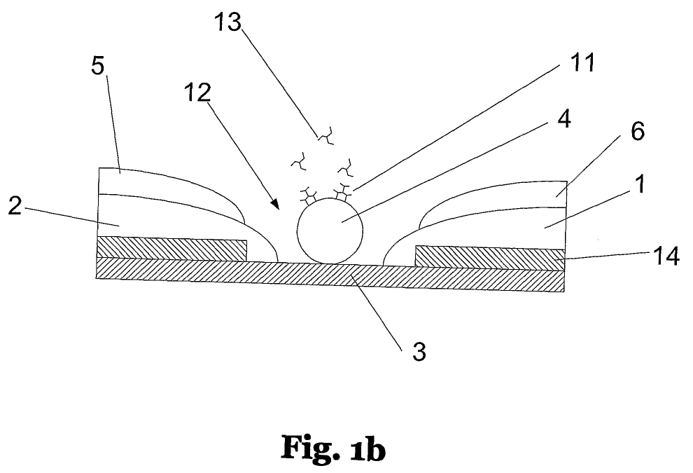

The sensing device 20 is part of a system 200 measuring and analyzing of measurement data; which is schematically illustrated in FIG. 2. The system 200 comprise the sensing chip 20 preferably located in a holding structure 210 for convenient change of sensing device 20 in order to test different substances or samples. The holding structure may be of a quick release type for quick and easy change of sensing chip 20. The sensing device 201 is electrically connected 208 to an electronic control system 203 which is adapted to provide measurement control signals and preprocess signals to appropriate format for digitization of the signals. The electronic control system 203 may comprise a dedicated control system with all electronics and communication built into one device or may comprise a combination of dedicated devices and commercially available electronic control and preprocessing devices. The electronic control system 203 may be connected 207 to a computational device 202 for controlling the measurements and analysis of obtained signals. The computational device may communicate with digital communication links and/or analogue links. With digital links is meant any suitable type of communication operating with digital data, such as direct digital links using dedicated digital I/O interfaces, Ethernet, serial (e.g. according to the standards RS232 or RS485) or parallel communication (e.g. Centronics or GPIB/HPIB (General Purpose Interface Bus/Hewlett Packard Interface Bus)), or according to wireless standards such as Bluetooth or WLAN (Wireless Local Area Network) protocols, e.g. according to IEEE 802.11, 802.15, and 802.16 standards families. The person skilled in the art should appreciate that other communication protocols may be used for this communication. With analogue links is meant A/D (analogue to digital) or D/A (digital to analogue) converters. The system may further comprise a pump 204 with a reservoir 209 and tubing 205, 206 for input and return of substances to the sensing device 201. The pump 204 is not necessary in all applications of the present invention, for instance when measuring the presence of substances in air, the sensing device 201 may be presented to the ambient air directly thus allowing the air into contact with the sensing device 201.

The entire system may be incorporated into one single device box such as a desktop instrument or even a portable instrument that can be used where ever it is of use, for instance at an airport for detecting small traces of explosives, gunpowder, or drugs in air that can help security and/or drug enforcement personal in their search for explosives, weapons and/or drugs.

### Processor Design

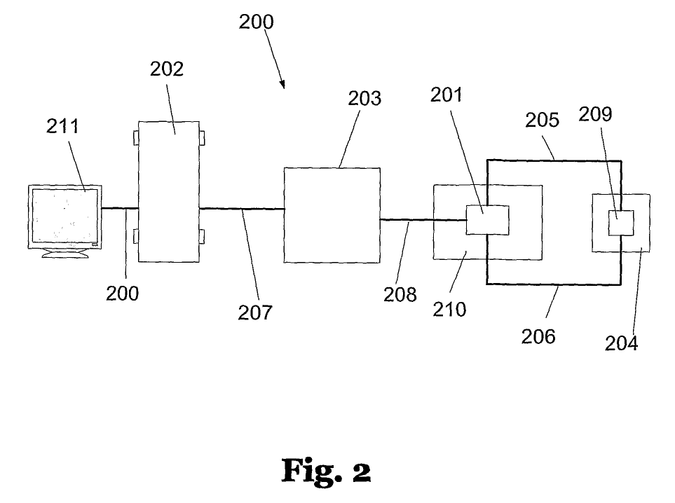

The present invention makes use of different types of signal analysis to determine the presence and amount of substances under detection. For this purpose a computational device 300 is used. This is schematically illustrated in FIG. 3 as a block diagram. The computational device 300 comprise a computational unit 301, one or several memory units 302, 302′, a communication unit 303, a pre processing unit 304, a measurement interface 306 to the sensing device, and a communication interface 305 to external equipment.

The computational unit 301 may comprise for instance a microprocessor wherein software operates signal analysis and controls user interface signals (both input and output signals), the computational unit 301 may store data in a memory unit 302, 302′ which may be volatile or non-volatile in its configuration, for instance RAM or hard drive memory units. The communication interface 303 communicates with external equipment for instance other computational devices such as personal computers in a network using Ethernet or other known communication protocols. The pre processing unit 304 may comprise a digital signal processor or A/D-D/A unit for controlling the measurement and receiving measurement signals. The measurement interface 306 may comprise an interface bus with one or a plurality of signal connectors directly or indirectly connected to the sensing device 201. Optionally the interface bus may comprise a communication interface for communicating with a processor located in an electronic control device 203 and communicating using any suitable protocol, for instance Ethernet or similar IP (Internet Protocol) based protocols.

The software and/or pre processing unit may comprise methods for signal analysis and signal processing, for instance averaging, normalizing, feature detection, slope detection, parameter detection, spectroscopy operation, filtering and other simple or advanced signal processing algorithms. The software and/or pre processing unit may also comprise measurement control such as controlling output signals to the sensing device 201 (e.g. voltage sweeps (for IV-curves), controlling valves in a fluidic pump system or controlling positions of gates and similar electromechanical devices. It can also control external measurement instrumentation, such as parameter setup and instrumentation configuration, triggering measurements and output signal generation.

In a further development of the sensing device 20, some intelligent functions may be incorporated onto the sensing device, for instance a pre processing unit and/or a buffer memory in order to handle signals from the electrode pairs 1, 2. For instance acquiring a large number of signals may benefit substantially with such a local intelligent functionality, increasing the real time characteristics of the system. The sensing device 20 is thus adapted to acquire signals from the electrode pairs 1, 2, either in parallel or in series into the memory directly or indirectly through the pre processing device.

### Gap Formation

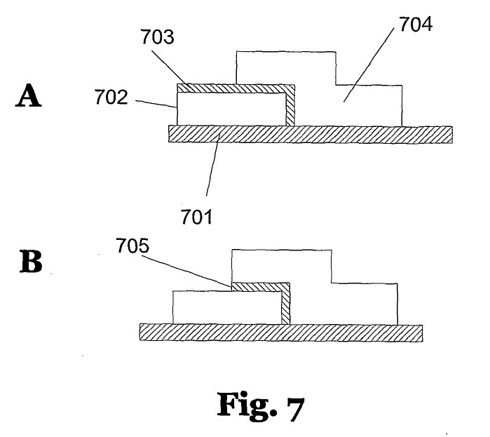

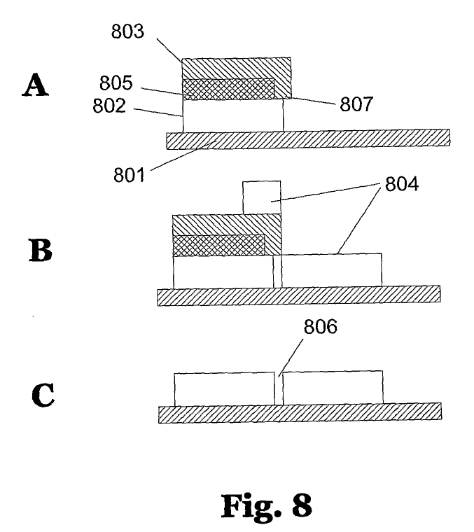

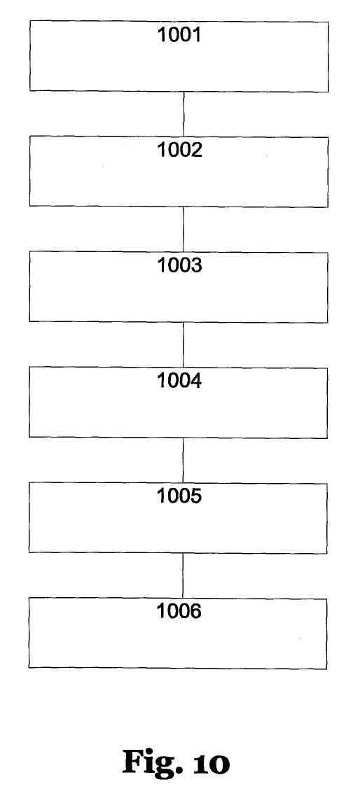

The fabrication of such a self-assembling SET requires electrodes separated by a gap small enough to trap a nanoparticle smaller than 10 nm, which is a challenging task. One essential feature in the production of the sensing device 20 is the gap 12 between the electrodes. This should preferably be well defined and reproducible in order to position the activation object suitably. There are several different methods of producing this gap during the manufacturing process:
1. Electron beam lithography can be combined with ion beam etching. The electrode resist pattern is made with electron beam lithography. A bottom sticking layer is evaporated perpendicularly to the sample. A gold film is evaporated with an angle to cover the gap with gold. Then another preferably hard and isolating layer e.g. SiO₂, is evaporated perpendicular to the sample. The sample is dry etched with ions, either perpendicular to the surface or with a small angle from the surface normal, until a gap with desired size is achieved.
2. To use short circuited gold electrodes and open the electrodes with gold etch while measuring the current through the electrodes. It is then possible to stop the wet etching just when the current decreases rapidly.
3. To use grains formation during evaporation in the gap between the electrodes. These grains could be bridged together by one or more gold particles attached by linking molecules. The rate of evaporation can be tuned in order to optimize the grain formation.
4. Electrodes can be separated by a thin insulating layer (or conducting layer which can be removed later in the process). Then the separation between the electrodes is defined by the thickness of the insulating layer. First one of the electrodes is fabricated. Then an insulating material that covers the first electrode partly or fully is fabricated. Then the second electrode is made so that it contacts the insulating layer. When the insulating layer is removed there is a defined gap (equal to the thickness of the insulating layer) between the two electrodes. The insulating layer can also be partially removed and thereby also allow for SET fabrication.
5. A two-layer resist system of a 140 nm thick lift-off bottom resist layer and a 60 nm thick PMMA e-beam top resist layer may be used. In the top resist layer a mask for a 50 nm gap may be defined with electron-beam lithography. To obtain gaps smaller than ten nanometers angle evaporation may be used by tilting the sample, through an axis perpendicular to the electrodes, at two different angles during metal evaporation. In this way the gap size is controlled by the tilting angle.
6. The radial distribution of energy in the resist due to e-beam exposure has been calculated numerically and can to a first approximation be estimated by a Gaussian distribution. By letting two beams overlap in the gap region the effective gap can be adjusted with the intensity of the beams and with the inter distance of the beams. If overexposure conditions are used the effective gap will be smaller then the intended distance. After development only the parts of resist that received a dose lower than a certain threshold value, Qt, will remain, forming the mask separating the two electrodes during evaporation. With increasing development time Qt decreases. Thus the gap size can be adjusted not only by the exposure conditions but also by the development time of the resist layer. In this way it is possible to compensate for variations in the exposure conditions, i.e. beam size, from exposure to exposure by choosing an appropriate development time. After each exposure gold may be evaporated on a series of test chips, developed for different development times of the top resist layer. By studying the size of the gaps for the different development times the optimal development time may be calculated.
7. An alternative gap formation which is reproducible is a so called self-aligned lithography (SAL). This is based on a gap formation alternative 4 (sacrificial layer method) mentioned earlier in this document. The sacrificial layer method is illustrated in FIG. 7, wherein a first metal electrode 702 is evaporated onto a surface 701 (directly or indirectly) and an oxidized aluminum layer 703 is formed on the first electrode 702. A second metal electrode 704 is formed on the surface 701 and at least part of the oxidized aluminum layer 703. In a final step part of the oxidized aluminum layer 703 is removed and a gap 705 is formed. FIG. 8 illustrates the SAL process in more detail. SAL makes use of the fact that when a metal is oxidized in will tend to increase the volume. The electrodes 1, 2 are fabricated one at a time and a layer of aluminum 805 is formed on a first fabricated electrode 802 onto a surface 801. This aluminum layer 805 is oxidized forming an aluminum oxide layer 803 and since the aluminum oxide layer expands in volume it will form an over hang 807 over outside the first electrode 802. In a subsequent step a second metal electrode 804 is formed on the surface and part of the aluminum oxide layer. In a final step the electrode material on top of the aluminum oxide layer, the aluminum oxide layer 803, the aluminum layer 805 are removed using different techniques a gap 806 will form between the two electrodes. This gives a highly reproducible and accurate gap 806, since the oxidization of the aluminum layer is a well controlled process both within the same wafer and between wafer to wafer. This is illustrated in FIG. 10 with method steps 1001 to 1006.

### Fabrication of Sensing Devices in Detail

In the following an example of fabrication of sensing devices 20 will be described; however other fabrication processes may be used.

The entire fabrication comprises five different parts:
Glass mask fabrication
Photolithography
Dicing
SiO₂ coating
Electron beam lithography

To be able to make electrical measurements on a nanoscale electronic device, such as a SET, it has to be contacted to the macroworld. The contact to the chip is made by making relatively large contact pads connected to the electrodes forming the nanogap. The contact pads are defined by photolithography. In the same step, alignment marks for the second step of photolithography and for the electron-beam lithography are defined.

A number of pairs of electrodes may be centered on 9×5 mm chips, and 72 chips may be exposed on a 3″ wafer. The width of the chips may be adjusted to a fluidic system that would be attached on top of the chip. The size of the chips simplifies the fabrication process since they are easier to handle with tweezers. The gold pads 8, created with a gold mask, may be covered in silicon dioxide. A special mask for this purpose may be created. The mask for SiO₂ covers the gold pads but leaves the ends exposed; one end for connection to the probe and the other for connection to the small electrodes.

A square 4″ mask of chromium coated soda glass may be used as surface onto which the resist Shipley UV-5 may be spun at 2000 rpm for 1 min and baked on hot plate at 130° C. for 10 min. The resist may be thereafter exposed in a high-resolution EBL system, JBX-9300FS, and immediately baked at 130° C. for 10 min. If the mask is not post-baked after exposure the patterned resist will degenerate if it is exposed to air. The resist may be developed in MF-24A, rinsed in water and carefully blow-dried. The mask may be descummed with oxygen plasma (50 W, 30 s) and placed in a container with Balzers Chrome Etch #4 until the desired pattern became transparent. The etching may be stopped by placing the mask in DI-water. Finally the resist may be removed in a bath with 45° C. hot remover 1165. The mask may be cleaned with IPA and DI-water.

A 3″ oxidized silicon wafer may be placed in acetone in an ultra sonic bath for 2 min at 100% effect. To remove the acetone the wafer may be rinsed in IPA and blow-dried with N₂. Further cleaning may be made by reactive ion etching (RIE) using 50 W oxygen plasma at 250 mTorr. The wafer may be spun with two resist layers. The bottom layer consisted of a non-photosensitive resist, LOL2000 (Lift Off Layer 2000) which may be spun at 3000 rpm for 1 min and post baked at 140° C. for 5 min. The second layer may be made by photo resist Shipley 1813 (s-1813), which may be spun at 4000 rpm during 1 min and baked at 90° C. for 3 min. It is also possible to use only the layer of S-1813 for the photolithography. Both approaches have advantages. When using a single layer the pattern is more robust and not sensitive to mechanical pressure as N₂ drying. However sometimes the lift off can be very difficult when using a single layer. This is because the evaporated film might cover the sidewalls of the resist layer connecting the pattern with the part of the film that should be removed. Which approach is most convenient depends on the geometry of the pattern. For patterns with many details with dimensions of a few micrometers it is generally preferable to use a single layer. To transfer the pattern from the mask, a Karl Süss mask aligner (MJB2) may be used. The wafer may be aligned under the mask and tightly pressed against the mask until an interference pattern became visible. This ensures good contact between the wafer and the mask which is a requirement for a good exposure. During 12 seconds the wafer may be exposed for light of wavelength 400 nm at an intensity of 10 mW/cm². The resist layers were developed in MF319 for 20 s. During development of the resist layers the exposed areas of s-1813 are dissolved. Because LOL2000 is non-photosensitive the entire layer is equally solvable. When the developer has dissolved the structures exposed in the top layer it will start to dissolve the bottom layer under the edges of the top layer producing an undercut. By changing the post bake temperature it is possible to regulate the extent of the undercut. The development may be stopped by placing the wafer in DI-water. The water may be with some difficulty removed by careful blow drying. A more careless N₂ drying caused deformations of tiny resist patterns. Unwanted resist rests may be removed by oxygen plasma for 30 s at 50 W. Evaporation of 10 nm Ti and 80 nm Au, with deposition rates of 0.1 nm/s and 0.2 nm/s respectively, may be done in AVAC HVC600. Whenever evaporation is mentioned it is evaporation with an electron gun heating the source and the pressure is around 1·10-6 mbar. The rates for Ti and Au are the same throughout the process. The lift off may be made in acetone at room temperature in ultrasonic agitation at 40% effect for 2 min. Since LOL2000 does not completely dissolve in acetone, the last traces of LOL2000 were removed in MF319. The wafer may be rinsed in isopropanol and de-ionized water.

Before dicing, the wafer may be spin coated with a few hundred nanometer protective layer of copolymer and baked on hotplate for 3 min. In order to be able to break the wafer into chips after processing, the backside of the wafer may be pre cut. Initial cuts were made from the front side and used as guidelines for the backside cutting. The cuts on the backside were about 100 μm deep and the thickness of the wafer may be around 350 μm. When the dicing may be finished the wafer may be cleaned in acetone, for removal of the spin coated copolymer layer.

The SiO₂ mask may be used to cover the large gold pads. The alignment, which may be not crucial for the gold pattern, is here of great importance since the end of the pads, used for connection to the small electrodes, must not be covered with SiO₂. To facilitate the alignment, alignment marks in the gold mask and the SiO₂ mask were made. The photolithography may be carried out with the same resists and parameters as described above.

A SiO₂ layer with a thickness of 50 nm may be evaporated at a rate of 0.1 nm/s. The evaporation rate for SiO₂ may be less stable than for metals. It is important to heat the SiO₂ source slowly over a large area in order to avoid large internal stresses. The evaporation rate became more stable when a large area of the source is heated.

For electron-beam lithography a resist system of two layers may be used in order to obtain an undercut necessary for future angle evaporation. As bottom layer 10% Copolymer (8.5% methacrylic acid in PMMA) in ethyl lactate may be used. The short name for this Copolymer is MMA (8.5 mM) EL10. Since structures down to the limit of the resolution of EBL are to be fabricated a very high contrast resist may be needed. The resist used for the top layer may be ZEP520A, which has a similar resolution as PMMA. The sensitivity, Q, is about 70 μC/cm2 which is a lower value than for PMMA.

The wafer may be spin coated with MMA (8.5 MAA) EL10 at 5000 rpm during 1 min and baked on hotplate at 180° C. for 10 min. ZEP520A may be spun on top at 5000 rpm for 1 min and baked at 180° C. for 10 min.

Without delay the wafer may be placed in a 3″ cassette and loaded into the high resolution EBL system, JBX-9300FS. It is important to align the wafer carefully when placing it in the cassette in order to avoid too large rotations since the system can not compensate for large errors of this kind. In order to achieve high resolution a low current of 300 pA is used. This low current should correspond to a beam size below 10 nm. In order to minimize the beam size, high requirements of elimination of astigmatism and focus errors were set. The selected dose may be 200 μC/cm². The entire wafer may be exposed with a number of electrodes (e.g. 2×8 as shown in FIG. 1a) per chip and 72 chips per wafer.

After exposure, four chips were cut from the wafer. P-xylene may be used as developer for the top resist layer and ECA:Ethanol 1:5 may be used to dissolve the bottom resist layer. At first one chip may be developed in P-xylene for 90 s and quickly dipped in IPA to remove P-xylene and possible resist rests. The chip may be immediately developed in ECA:Ethanol 1:5 for 150 s, dipped in IPA and put in DI-water for a few seconds until the IPA is dissolved. When the chip is removed from the water no drying is necessary. The surface of the chip is so hydrophobic that no visible water will be left on the surface if it is removed slowly from the water. This treatment is gentler than N₂ drying which might be of importance if the resist bridge in the gap is very small. The three other chips were developed in the same way but with different developing times in P-xylene, (120 S, 150 s and 180 s). Between different exposures there is always some variation. However this variation could be compensated by slightly changing the developing time. Since the contrast is very high one may argue that it should not be possible to manipulate the gap size between the electrodes by changing the developing time. This is true, but when the difference in gap size is in the order of about ten nanometers it should be possible especially since the gap certainly has received some proximity radiation. Another developer (O-xylene) may be used. Compared with P-xylene, it is harder to make small gaps with O-xylene.

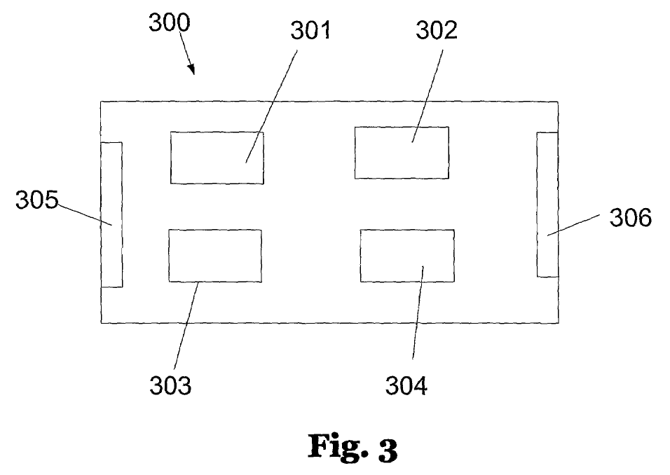

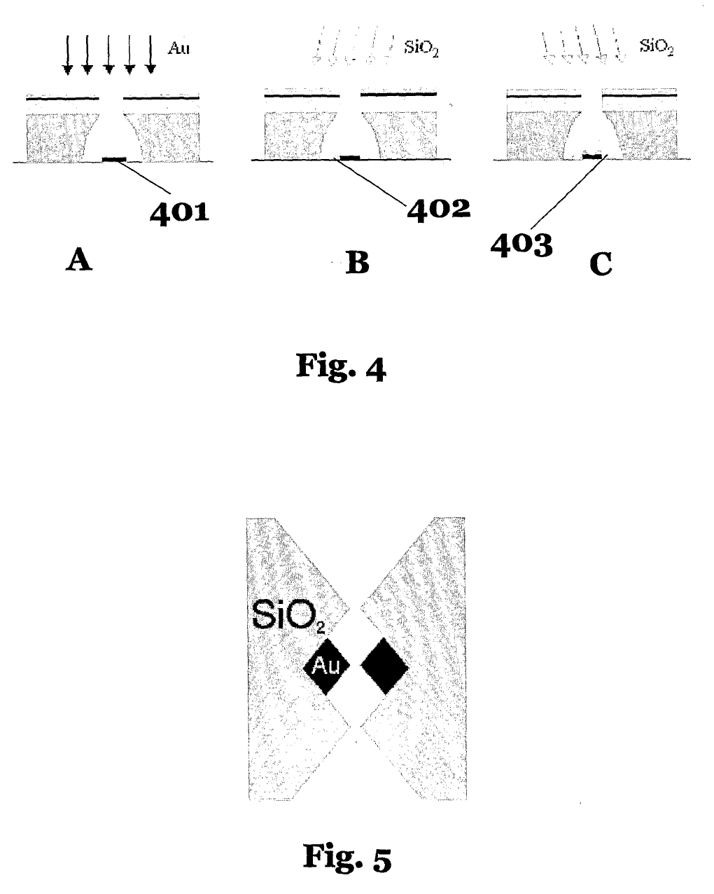

The following will describe one way of forming the insulating layer; however, it should be understood by the person skilled in the art that other ways exist As an example SiO₂ will used as insulating layer, however, other materials may be used such as for instance titanium oxide, aluminium oxide, chromium oxide, iron oxide, beryllium oxide, ceramics, polystyrene or Teflon. It should be understood by the person skilled in the art that the fabrication method might be slightly changed depending on material used. Four developed chips were placed in AVAC HC600 and a 10 nm Ti film may be evaporated. On top of the Ti film a 25 nm thick gold film may be evaporated. Lift off may be performed in Acetone at 40° C. In order to decide which developing time produced the best results a SEM of type JEOL-JSM 6301F may be used to characterize the four chips. The developing time for the chip with the best result may be chosen for the rest of the wafer. After the rest of the wafer is properly developed it may be placed on a tiltable surface holder in AVAC HC600. A 10 nm Ti film may be evaporated as adhesion layer. On top a gold film with a thickness of 25 nm may be evaporated. Now the surface may be tilted 10°, and a 4 nm Ti film may be evaporated. The shutter may be closed while the surface may be tilted to −10°, and another 4 nm Ti film may be evaporated. At this angle 20 nm SiO2 may be evaporated. The surface may be tilted back to 100 and another 20 nm SiO2 may be evaporated. This covered the electrodes with SiO2 except for the tips. The evaporation process is schematically shown in FIG. 4, which is a side view of the evaporation process. In A, gold is evaporated onto the chip forming an island 401 and in B SiO2 is evaporated from one angle and in C, from another angle forming two different insulating layers 402, 403 around the gold island. One example of how the SiO2 areas may be placed is shown in FIG. 5 which is a top view of the same situation as described in relation to FIG. 4.

### Binding Chemistry of Activation Object 4

The biologically active site of the sensor is the activation object (4) such as for example a functionalized gold nanoparticle. Properties like for example surface energy, surface chemistry, dielectric properties and surface charge are important when developing devices with a biological interface. For biosensors, it is obvious that the biological interface must be provided with the ability to perform biological recognition, achieved through immobilisation of biologically active agents like DNA or, with increasing occurrence, proteins like antibodies. In order to perform this immobilisation, the surface must provide appropriate “chemical handles” depending on which immobilisation methodology that is chosen.

However, surface properties are not only important for the sensor's specific interactions; they also have great impact on the sensor's unspecific interactions. Surface wettability and charge also influence the amount and type of proteins that adsorbs on surfaces from biological plasma. The same is valid for the surface mobility and water binding capacity, which also affects the protein binding behaviour. If the origin of the sensor signal is based on an electrochemical reaction, the catalytic activity of the surface also is an important parameter.

### Gold Nanoparticle Preparation

Nanoparticle preparations involve the use of a stabilising agent, which can associate with the particle surface and provide the particle with some properties that make it stay in solution. Without such a stabilizing agent, the nanoparticles will aggregate and precipitate. Two main routes for synthesis of stabilized nanoparticles especially well suited for the construction of devices and nanostructures can be used. Both methods depend on reduction of gold (III) derivates, commonly the salt AuCl₄⁻. The choice of reductive or stabilizing agent however differs as do the nature of the phase in which the particles are synthesized. In this context, the stabilized gold nanoparticles, are abbreviated AuNPs.

The first method used for AuNP preparation is reduction of AuCl₄⁻ with citrate as reducing agent in aqueous solution, well known to the person skilled in the art. This method yields roughly spherical particles with a narrow size distribution that can essentially be controlled by the initial citrate to AuCl₄⁻ ratio, where higher ratios give smaller particles. This is however only valid for larger particles. In order to prepare the smallest particles, i.e. around 5 nm or less, the citrate reduction method can be used if tannic acid is added as an extra reductive agent. In the case of citrate mediated particle preparation, the citrate does not only act as reductive agent, but also as stabilising agent. The citrate adheres loosely to the gold core, providing the AuNPs with a negative net charge. Thus the particles in the solution become stabilised due to the electrostatic repulsion between neighbouring particles and are prevented from aggregation. Because of this, these nanoparticle solutions are very sensitive to contamination, especially by salts, which eventually will make the particle aggregate and precipitate.

In the second method for AuNP preparation the reduction of AuCl₄⁻ is not performed in aqueous solution, but the salt is transferred to an organic solvent using a transfer agent. Once in the organic solvent, AuCl₄⁻ is reduced by addition of a reducing agent, commonly NaBH₄. This is done in the presence of long-chain alkane thiols, which bind to the AuNPs and stabilise them due to sterical interaction between neighbouring alkane-coated particles. In contrast to particles prepared by citrate reduction, in this case the reductive agent and the stabilising agent are different.

An advantage of the alkane-thiol/NaBH₄ method is that it yields particles that are thermally and air stable and which can be easily transferred between different organic solvents. Further, by altering the thiol to AuCl₄⁻ ratio in the preparation, particles with narrow size distributions having mean core diameters ranging between 1.5 and 5.2 nm can be produced. The core size decreases with increasing thiol to gold ratio. Finally, these kinds of alkane-thiol stabilised particles constitute a particle analogue to the flat surface self-assembled monolayers SAMs, discussed below.

A way to attain surfaces, including nanoparticles, with especially well defined properties is to modify them with so-called self-assembled monolayers, SAMs, i.e. molecules deposit spontaneously on a surface whereupon a more or less ordered molecular film is formed. Among the organosulfuric compounds, different alkanethiols can be used for the formation of SAMs on gold surfaces. The dominant factor in the choosing process is the formation of the very strong (adsorption energy 145-188 kJ/mol) thiolate-bond to the gold surface. Such SAMs are characterised by densely packed monolayers where the alkyl chains order themselves in a slightly tilted, all trans configuration that allows optimal lateral interaction between the molecules. However, the exact conformation strongly depends on the length of the alkyl chains. For chains with less than 12 carbon atoms, the SAM exhibits an increasing degree of unordered structure at the top of the monolayer (all trans-gauche) and for chains with less than eight carbon atoms, the structure is totally unordered (gauche). Due to the alkyl chains the particles become stabilized sterically and the surface charge does not have to be considered. This allows more complex structures to be created and functionality (discussed below) can be added already during particle synthesis.

The procedure for preparation of thiol SAMs is straightforward, even though special caution is necessary considering cleanliness in order to avoid contamination of the gold surfaces. The gold surfaces are, subsequent to extensive cleaning, immersed in thiol solution. Which solvent to use depends on the properties of the thiols, like the carbon chain length. Normally ultra pure ethanol is an appropriate solvent for thiols having up to 18 methylene units. For longer thiols an organic solvent, e.g. hexane has to be used. The smallest thiols like mercaptopropionic acid or cysteine can be deposited from aqueous solvent. Besides the solvent, also the temperature, immersion time and quality of the gold surface are important parameters determining the quality of the SAM.

### Self-Assembled Monolayers as Functional Surfaces

The versatility of SAMs lies in the possibility to use thiols with different head groups without affecting the underlying ordered structure. By choosing an appropriate terminal group, SAMs can be prepared displaying almost any desired surface property regarding for example wettability or the possibility of further protein immobilisation, i.e. functionalization. Surfaces can be created with long chain alkanethiols displaying for example either hydrophobic methyl head groups or hydrophilic hydroxyl groups in order to achieve protein adsorption. Surfaces can be constructed with terminal carboxyl groups or biotin groups allowing protein coupling through carbodiimide chemistry, see below, or specific biotin-Avidin interaction.

Usually only one type of thiol is used for the monolayer formation. However, SAMs consisting of more than one species, so-called mixed SAMs can be prepared. The motive for this might be a requirement of multiple functionalities or optimisation of the distance between for example protein anchoring points.

In order to perform a successful SET-assay, stabilized gold nanoparticles must not be too large, i.e. gold nanoparticles with a diameter of 20 nm or less shall be prepared, more preferably a diameter of less than 18 nm, more preferably a diameter of less than 16 nm, more preferably a diameter of less than 14 nm, more preferably a diameter of less than 12 nm, more preferably a diameter of less than 10 nm, more preferably a diameter of less than 8 nm, more preferably a diameter of less than 6 nm, and most preferably a diameter of less than 4 nm

A general comment regarding the preparation of AuNPs, valid for both methods presented, is that the preparation is a quite delicate procedure. Even though AuNPs are considered as the most stable metal nanoparticles, special precautions must always be taken to avoid their aggregation and precipitation. For example glassware must be cleaned thoroughly and chemicals must be handled very carefully and accurately. Further, slightest deviation in the preparation process might affect the outcome regarding particle size and size distribution. This holds for factors like temperature, different batch volumes, different sized glassware, different stirring and different rates for the addition of reductive agent.

### Example 1

### Fabrication and Size Separation of Citrate Stabilized Gold Nanoparticles

Gold nanoparticles were fabricated by tannic acid assisted citrate reduction of tetrachloroaurat (AuCl₄⁻). The size of the obtained gold nanoparticles depends on the amount of tannic acid added, more tannic acid giving smaller sized particles. AuNPs were prepared in different batches with a mean size of 14 nm and 5 nm respectively, according to the following protocol:

All glassware used was extensively washed with Helmanex™ and extensively rinsed with water (MilliQ ultra-pure distilled water>18.2 μl, MilliPore System). In order to make 100 ml of raw AuNP solution, two stock solutions were prepared: I, 80 ml water was mixed with 1 ml aqueous AuCl₄⁻ (1% in water, SigmaAldrich) and II, 16 ml water was mixed with 4 ml tri-sodium citrate (1% in water, SigmaAldrich) and either 0.025 ml or 2.50 ml tannic acid (1% in water, SigmaAldrich) for large and small particles respectively. For the smaller particles, also 2.50 ml of K₂CO₃ (25 mM in water, SigmaAldrich) was added to the second solution for pH adjustment. Both solutions were heated to 60° C. whereupon the solutions were mixed under continuous stirring.

After mixing, the solution changed colour from slightly yellow to violet and then to red, a procedure that took about one hour for the larger particles but only a couple of seconds for the smaller particles. When the red colour was completely developed, the AuNP solution was heated to 95° C., whereupon the solutions were cooled on ice. The particles were stored at room temperature or at 4° C.

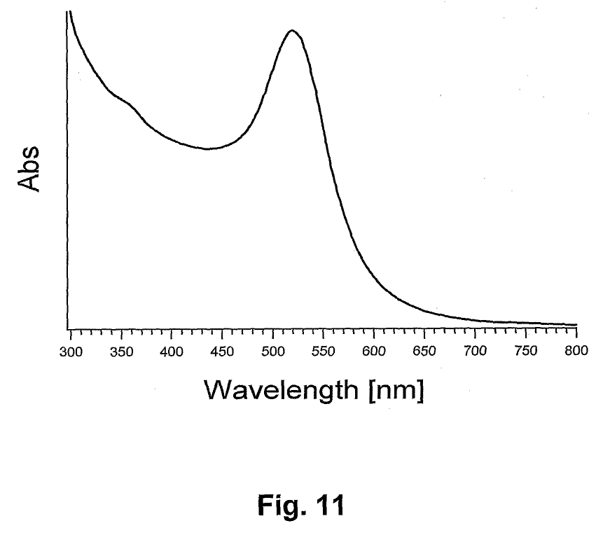

The obtained particle solutions, referred to as “raw” solutions, were characterised with spectrophotometry in the visible wavelength area. The observed resonance frequency or localised surface plasmon resonance (LSPR) is normally positioned around 520 nm for gold nanoparticles sized between 1 and 40 nm. The LSPR depends on the dielectric properties of the ambient medium, the particle size, particle shape, particle charge, temperature and the inter-particle distance. All these factors will cause a shift of the peak resonance either towards longer (red shift) or shorter (blue shift) wavelengths. FIG. 11 shows the visible spectra for raw 14 nm AuNP solution. The dominating feature is the strong symmetric absorption peak at exactly 520 nm due to localised surface plasmon resonance of the particles giving rise to the solution's ruby red colour. Besides the LSPR peak, the solution also absorbs strongly for shorter wavelengths near the UV area. This corresponds to a high background of ultra-small particles and AuCl₄⁻ in solution. The small perturbation of the curve at approximately 360 nm corresponds to absorption by tannic acid. In order to remove excess tannic acid and AuCl₄⁻, and to separate too small and too large particles from the AuNP solutions, different centrifugation techniques were employed. For the larger AuNPs (14 nm), an ordinary cooled bench-top centrifuge could be used to pellet the nanoparticles by centrifugation of the raw AuNP solution for 30 minutes at 7000 g. After carefully removing the supernatant, the pellet was diluted in 1% citrate solution whereupon the centrifugation procedure was repeated once or twice in order to wash the particles. After the last centrifugation, the particles were diluted to gain a desired concentration, i.e. colour. The particle solution obtained was examined with spectrophotometry.

The smaller particles (5 nm) are too small to pellet using a bench-top centrifuge, but high-speed ultra-centrifugation must be employed. A two-step ultra-centrifugation procedure was employed using a SW40 rotor (40 000 rpm) and Ultra Clear™ Tubes, 14×95 mm, Beckman tubes. Six centrifugation tubes were filled with raw AuNP solution, totally about 85 ml.

The first centrifugation was performed at 210 000 g at 4° C. for 30 minutes, whereupon the red supernatant was transferred to new tubes. The formed pellet, containing larger and aggregated particles, was discarded. The tubes with red supernatant, containing particles of smaller and desirable size, were centrifuged in a second step at 225 000 g at 4° C. for 75 minutes. It was noted that a shorter centrifugation time, i.e. 45 minutes, did not give any pellet. After the second centrifugation step, a red pellet had formed in the bottom of the tubes, whereas the supernatant was yellowish. After discarding the supernatant, the pellet could be dissolved in 1% citrate solution, yielding a high concentrated AuNP solution. The AuNP solution was characterised using spectrophotometry.

Exclusion of the first centrifugation step, i.e. direct centrifugation of the raw solution at 225 000 g led to irreversible aggregation of the particles.

It should be noted that the centrifugation procedure generally is very sensitive. Any deviations in the characteristics of the raw solutions, e.g. colour, led to centrifugation-induced aggregation. Further, the temperature seemed to be an important factor. Large 14 nm particles centrifuged 3×30 minutes in a bench-top centrifuge at room temperature aggregated, whereas the same procedure performed with a cooled bench-top centrifuge did not lead to aggregation. Dilution of the pellet with water instead of 1% citrate solution also made the particle solutions less stable. Whereas the AuNPs in citrate solution could be centrifuged and dissolved repeatedly, the AuNPs in water did not manage more than two centrifugations without aggregating.

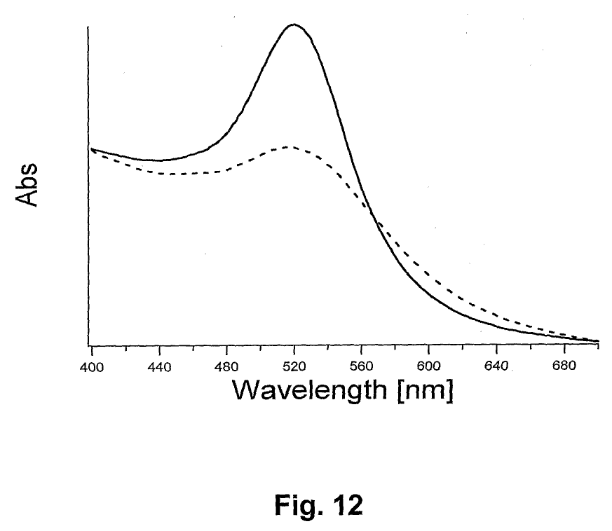

FIG. 12 shows and highlights the differences between visible spectra obtained for the larger and smaller particles after centrifugation. For the larger particles, the LSPR absorption peak is strong and distinct, which is characteristic for larger particles. The peak absorption maximum is situated at 520 nm, which corresponds to citrate-stabilised particles in water with a size larger than 10 nm but smaller than 20 nm. For the smaller particles, the LSPR peak is significantly dampened and broadened. Close examination of the peak wavelengths shows that the peak maximum is slightly shifted from 520 nm to 517 nm for the smaller particles. This corresponds well to what is expected from theory, regarding small particles. To the eye, both solutions appear clearly red. However, whereas the solution with large particles may be defined as deep ruby-red, the solution with smaller particles is only red with a tint of brown.

### Gold Nanoparticle Functionalisation

The synthesis of gold nanoparticles with specific surface functionality is desirable for many purposes. The main objects are nanoparticle handling (solubility and stability in different environments), the formation of macromolecular conjugates and construction of functional architectures in the context of nanotechnology. Nanoparticles can be functionalized with the binding any kind of binding structure to its surface. Functionalization can for example be achieved by binding single ions, atoms, low-molecular compounds, nucleotides, DNA-fragments, amino acids, peptides or proteins for the construction of functional structures or detection of chemical reactions. AuNPs can be conjugated with biologically active species, such as for example low-molecular compounds, DNA-fragments, DNA-sequences, amino acids, peptides, proteins, receptors, antibodies, enzymes to perform a biologically active reactions, molecular imprinted polymers. The functionalisation of the nanoparticles can be accomplished following two general routes: either is the functionality provided already during the AuNP synthesis (as discussed above), or added to the particles after the synthesis trough some exchange reaction where the stabilising ligand layer is exchanged.

For the case of citrate stabilised AuNPs in aqueous solution, the second method, exchange mediated functionalisation, dominates. Since the citrate shell surrounding the nanoparticles is quite loose, it can easily be exchanged. For example, conjugates between gold nanoparticles and certain peptides, proteins, enzymes or antibodies, can be prepared by physisorption of the proteins directly onto the particle surfaces for use within protein chemistry applications. AuNPs can be conjugated with proteins such as for example Avidin in order to perform specific immobilisation of the nanoparticles to a biotin-functionalised surface. Also AuNPs can be functionalized with low-molecular compounds, to primarily for the purpose of constructing functional architectures. Stabilized gold nanoparticles form strong covalent or covalent-like bonds to molecular compounds having thiol, sulphide, amine, cyanide, diphenylphosphine or pyridine functional groups.

Amines, which normally form only weak bonds and chemically unstable monolayers on gold surfaces, bind almost as strong as thiol compounds to AuNPs. The process of exchanging citrate for other molecules means that the stabilising agent is removed from the nanoparticle surface. Because of this, it is important that the new ligand shell also can provide stability, usually by electrostatic repulsion. Since the molecular charge often is explicitly controlled by pH this also becomes an important parameter for controlling the stability of the AuNP solution. Further, if the new ligand molecules display more than one of the functional groups mentioned above, one may expect that the particles aggregate due to cross-linking.

The molecular species which can be used for this type of exchange-mediated functionalisation are typically water solvable ionic or zwitterionic compounds like mercaptopropionic acid and amino acids. For bifunctional amino acids, e.g. cysteine and lysine, they can work either as cross-linkers or stabilisers depending on the pH of the solution. Preferably the functionalising group is covalently bound to the AuNPs but may, however, also be electrostatically attached.

AuNPs can also be reacted with high molecular weight aminodextran. Each aminodextran can be functionalised with one biotin group, and by strictly controlling the reaction parameters, each gold nanoparticle is functionalised with one dextran chain and thus also with one biotin molecule. In this case, repulsive electric forces do not stabilise the nanoparticles in the solution, but the bulky dextran polymers stabilise the nanoparticles sterically. The same is valid for functionalisation of AuNPs with long-chain hydrophilic thiols like for example polyethylen glycol substituted alkane thiols.

### Example 2

### Avidin Functionalisation of Gold Nanoparticles

Avidin is a glycoprotein found in raw egg white. It combines stoichiometrically with biotin. The great affinity of Avidin for biotin, makes the system as a versatile platform for binding any biotinylated proteins such as antibodies or Fab-fragments for use for example in immunoassays, receptor and histochemical studies.

Large gold AuNPs (14 nm) prepared according to the procedure in Example 1 above were surface modified with Avidin by crude adsorption, according to a modified method as follows, which yields AuNPs completely covered with a monolayer of Avidin: Avidin (1 mg/ml in Tris, Sigma-Aldrich) and subsequently CaCl₂ (50 mM in water) was added to gold nanoparticles in an Eppendorf tube, rendering a final concentration of 0.05 mg/ml and 5 mM respectively. The CaCl₂ was added in order to prevent the Avidin coated particles from sticking to each other. In order to get rid of excess Avidin, the coated particles were centrifuged at 7 000 g at 4° C. for 30 minutes, whereupon the pellet was diluted in 5 mM CaCl₂ to desired volume. The CaCl₂ was added in order to prohibit the Avidin coated particles from attaching to each other. Addition of Avidin to the AuNP without subsequent addition of CaCl₂ led to slow, but spontaneous aggregation of the particles. The addition of Avidin to the surface of the gold nanoparticle will presumably affect the LSPR of the particle. Hence, the coating process could be monitored with spectrophotometry.

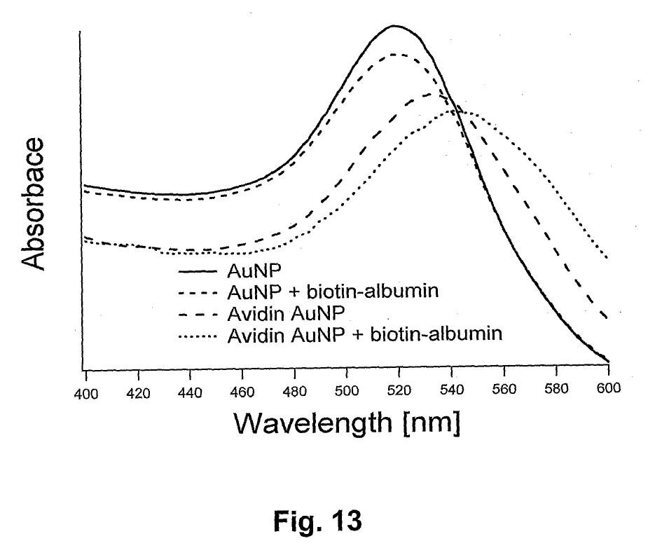

FIG. 13 shows visible spectra of the AuNPs before and after coating with Avidin. It can be seen that the coating process gave a shift in absorption peak maximum of approximately 14 nm as well as a slight broadening of the peak. According to numerous earlier studies, the absorption of a protein should render a red shift, and hence the adsorption process was probably successful.

In order to test whether the Avidin-functionalised particles exhibited biotin-binding ability, a small volume of biotin labelled bovine serum albumin (biotin-BSA, 1 mg/ml in Tris, Sigma-Aldrich) was added to cuvettes with Avidin coated AuNPs and uncoated reference AuNPs respectively, just before recording absorption spectra, rendering a biotin-BSA concentration of approximately 0.05 ng/ml. Since the biotin-BSA has eight biotin molecules per BSA it should be able to cross-link the Avidin-coated particles, i.e. cause aggregation. FIG. 13 shows the spectra obtained after biotin-BSA addition. It can be seen that whereas the biotin-BSA does not affect the reference particles at all, apart from a small dilution effect, the Avidin-coated particles suffer an additional 10 nm red shift as well as broadening of the absorption peak. This is a clear indication of biotin-BSA reacting with the Avidin functionalised AuNPs and making them aggregate.

The extent of the aggregation-induced LSPR shift might appear small compared to the shifts seen for salt-induced aggregation, which can have magnitudes of up to 100 nm. One must however remember that the effects of interparticle coupling on the LSPR decrease exponentially with the distance between the particles. Assuming that each protein (Avidin and BSA) is about five nanometres in diameter, the distance between two cross-linked particles will be about 15 nm, i.e. the same size-order as the particle diameter. Regarding this, it is not surprising that the protein-induced aggregation gives less red shift compared to for example salt-induced aggregation where the interparticle distance approaches zero.

In order to test the usability of the commercial SPR sensing system Biacore for detection of AuNP immobilisation, Avidin coated gold nanoparticles (14 nm) as described above, were immobilised on a gold surface functionalised with biotin-BSA. A plain gold surface (SIA Kit Au, Biacore) was cleaned in solution of 5:1:1 parts of water, hydrogen peroxide (30% v/v) and NH₄OH (25% v/v) at 80° C. for 5 minutes, whereupon it was rinsed thoroughly with water and immersed in a solution of biotin-BSA (0.1 mg/ml in PBS pH 5.5). After incubation over night, the surface was rinsed with water and dried with N₂, whereupon it was mounted onto a surface holder according to the instructions from the manufacturer.

The Biacore system used was a Biacore 2000, and HBS running buffer (Biacore AB) was used as running buffer. Avidin coated gold nanoparticles were injected in one flow channel at a flow rate of 10 μl/min during 10 minutes. In order to compare the signal obtained from AuNP binding with the signal from protein binding, Avidin (1 mg/ml in PBS pH 7.5) was injected in another flow channel at the same flow rate and time as for the AuNPs.

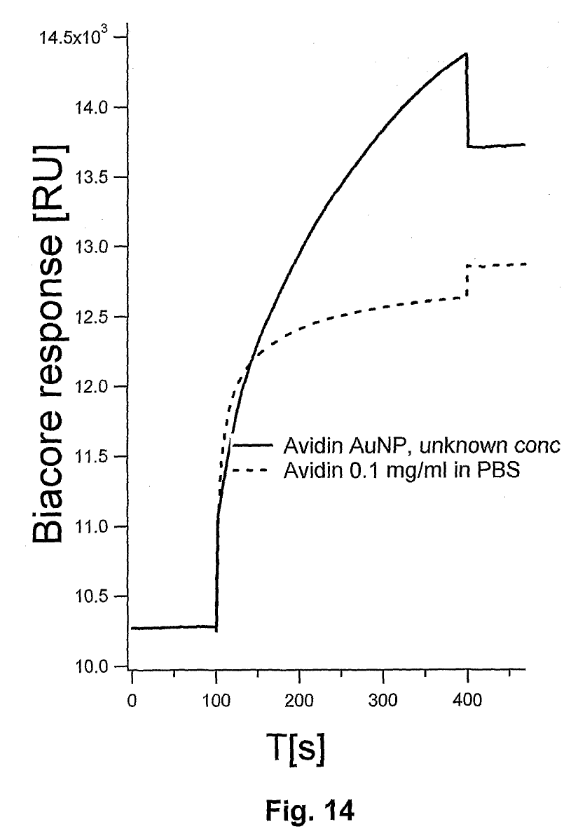

FIG. 14 displays the Biacore response curves obtained for five minutes injection of Avidin-AuNP solution, as well as reference Avidin solution of 0.1 mg/ml. The concentration of Avidin-coated AuNPs was very low, i.e. the solution appeared colourless to the eye. The reason for this was that most of the protein-coated particles had stuck to the walls of the Eppendorf tube where they were stored.

From the reference curve obtained for Avidin binding it is obvious that the biotin-BSA modified surface exhibited Avidin binding ability. The Avidin binding appears to take place quickly and reaches a saturation level after five minutes (the end of the injection), corresponding to 2567 RU of bound Avidin. During the same time, the binding of Avidin functionalised AuNPs results in an increased Biacore response of 3420 RU. This does not correspond to an extreme enhancement of the signal compared to Avidin binding, however one has to regard the fact that the binding of Avidin functionalised AuNPs is far from reaching the saturation level after the five-minute injection. The slow binding is due to both the low concentration of the Avidin-AuNP solution and the low diffusion constant for the large and heavy gold nanoparticles. Therefore, it is obvious that functionalisation of AuNPs with biologically active molecules is a powerful technique for SPR signal enhancement.

Also much diluted solutions of AuNPs yield a high SPR response as the gold nanoparticles bind to the surface. The mechanism behind the strong signal is that AuNPs have a large dielectric function and interaction between the propagating surface plasmon and the LSPR of the gold nanoparticles. Besides the shift in SPR angle presented as RU in the Biacore, the immobilised 14 nm AuNPs also induced dampening of the SPR signal, which could be detected as elevated reflectivity and broadening of the resonance dip (results not shown).

In this example Avidin is chosen as a model protein to illustrate how gold particle can become functionalized by an exchange mediated reaction. However, it shall be understood by the person skilled in the art, that a similar procedure can be applied for proteins with similar properties.

### Functionalisation of Gold Nanoparticles Through Alkanethiol or Cystine Coating

The other method for gold nanoparticle functionalisation, i.e. addition of functionality already during the particle synthesis, is primarily employed for thiol-stabilised particles (discussed above) prepared according to the phase-transfer method. Since the particles become stabilised sterically, the surface charge does not have to be considered, allowing more complex structures to be created. These nanoparticles can be made with multiple functionalities by using more than one thiol or by using asymmetrical disulfides having two distinct functional groups. This method is very versatile, for example the gold nanoparticles can be functionalized with single-stranded DNA-substituted alkanethiols.

Since the degree of order within the monolayer structure decreases with decreasing chain length, monolayers made from smaller thiols like mercaptopropionic acid or cysteine are not well structured. As the molecular organisation not always is considered to be the most important feature, but rather the surface functionality, using such SAMs might be a good idea. Cysteine (i) is of special interest since cysteine form monolayers on gold with both the amino groups and the carboxyl groups freely protruding away from the surface, which is advantageous since both amine and carboxyl groups constitute good handles for covalent coupling chemistry like carbodiimide or glutaraldehyde coupling of for example proteins.

The functionalisation of AuNPs directly by addition of cysteine to the particle solution is however not unproblematic. Mixing of particle solution (pH 5.5) with a relatively small amount of cysteine (10 mM in citrate buffer, pH 6.0) leads to slow but spontaneous aggregation after 30 minutes of mixing. This is due to a cysteine induced cross-linking of AuNPs as both the thiol group and the α-amine of the cysteine bound to different AuNPs. This process is pH-dependent and the α-amine can only bind AuNP surface when the carboxylate group of the cysteine is protonized, i.e. for low pH. For higher pH, the electrostatic repulsion between the citrate stabilised AuNPs and the negatively charged carboxylate would hinder the adjacent amine from binding. However, regarding the slow aggregation that occurred in our experiment at pH 5.5, where the carboxylates should be deprotonized, it is likely that also additional mechanisms are involved. Since cysteine is zwitterionic, at intermediate pH the net electrostatic repulsion between cysteine coated particles might be too low to stabilise the solution.

An alternative approach to achieve AuNPs with cysteine surface functionality can be achieved by adsorption of the symmetric disulfide cystine (ii), i.e. the disulfide counterpart to cysteine, to the AuNPs. Upon adsorption to a gold surface, the disulfide bridge of cystine breaks apart and the molecule becomes attached to the surface as two thiolate bound cysteine molecules.

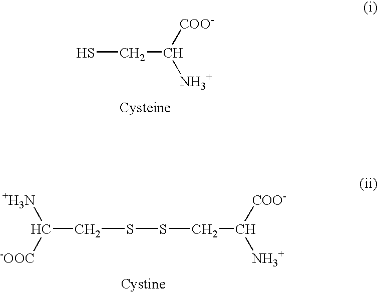

Monolayers can be formed from thiols or disulfides and in both cases thiolate bonds are formed. However, whereas thiols undergo oxidative adsorption, the adsorption of disulfides is reductive since the intermolecular S—S bridges have to be cleaved as an initial step. As a consequence, the kinetics for disulfide adsorption is different. Compared to cysteine adsorption, adsorption from cystine requires about 40% longer time for onolayer formation. The SAM formed is also less dense compared to the SAM formed rom cysteine. Preferably the cystine functionalized particle should have a mean diameter of less than 20 nm, more preferably a diameter of less than 18 nm, more preferably a diameter of less than 16 nm, more preferably a diameter of less than 14 nm, more preferably a diameter of less than 12 nm, more preferably a diameter of less than 10 nm, more preferably a diameter of less than 8 nm, more preferably a diameter of less than 6 nm, and most preferably a diameter of less than 4 nm.

### Example 3

### Cystine Coating of Gold Nanoparticles

Equal volumes of citrate stabilised AuNP (5 nm) solution prepared according to the procedure described above and saturated cystine solution were mixed and incubated in room temperature over night. The solvability of cystine in water is very low, i.e. only 53 mg/ml or 221 μM, why a saturated cystine solution was prepared for the functionalisation. As a reference, AuNP solution was also mixed with water and treated in the same way as the functionalised AuNPs. In order to remove excess cystine and citrate from the solution and enhance the particle concentration after functionalisation, the AuNP solution was loaded into centrifugation tubes (Ultra Clear™ Tubes, 14×95 mm, Beckman) and centrifuged at 225 000 g at 4° C. for 75 minutes. After centrifugation, the pellet was diluted to desired concentration with water. This method yields solutions that are stable, however sensitive. The solution with functionalised AuNPs could be centrifuged and the pellet fully redissolved in water without any aggregation. However, further centrifugation of the coated particles led to aggregation. The functionalised gold nanoparticles and the reference particles were characterised using spectrophotometry.

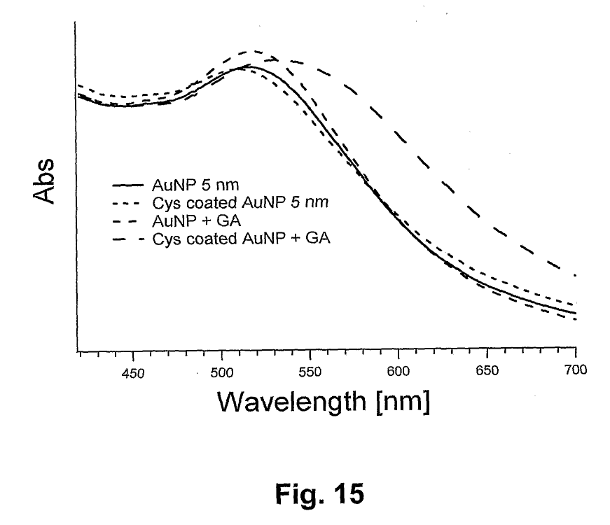

In FIG. 15 the shift of the LSPR peak maximum obtained for 5 nm AuNPs after adsorption of cystine, centrifugation and redissolution of the pellet in water can be seen. A significant blue shift from about 516-517 nm to 507-509 nm could be observed. The blue shift corresponds to a lowering of the refractive index around the gold nanoparticle. This shift indicates that something has happened at the surface of the AuNP and the fact that the shift is towards higher energies makes it feasible to exclude aggregation as the possible mechanism behind the shift.

The magnitude of the shift is quite large regarding the small size of the cysteine group compared to for example a protein. This reflects that strong thiolate bonds have been formed at the particle surface as well as the fact that the AuNPs are very small, i.e. only 5 nm in diameter, which gives rise to larger shifts compared to larger particles. The magnitude of the LSPR peak shift after cystine functionalisation gives a hint about the stability of the solution. Solutions having LSPR peak shifts only to 510 nm or higher do not remain stable over time, whereas solutions with longer shifts, i.e. 507-508 nm remain stable.

Since both thiol groups and amino groups are known to bind AuNPs, one possibility would be that both thiol groups and amino groups of the cysteine are coordinated to the gold surface. However, for the coupling chemistry to work, it is important that the amines are free and accessible. In order to test this, glutaraldehyde to a final concentration of 1% was added to cuvettes with cystine functionalised AuNPs and uncoated citrate stabilised AuNPs just before recording absorption spectra. If the amines were accessible at the surface of the AuNPs, the bi-functional glutaraldehyde would cross-link the particles, i.e. induce aggregation.

FIG. 15 shows the different spectra obtained. It is obvious that the addition of glutaraldehyde has very little impact on the reference particles, the LSPR peak red-shifts 1-2 nm and the absolute absorbance increases somewhat, probably due to the shift in solution refractive index upon addition of glutaraldehyde. For the cystine coated AuNPs however, the effect of glutaraldehyde addition is dramatic: the LSPR peak is red shifted from 509 nm to 531-533 nm, i.e. about 23 nm, and the peak is significantly broadened. The change of colour is very quick and clearly visible for the eye. This clear indication of aggregation implies that indeed glutaraldehyde mediated aggregation occurred and hence, that amines are accessible at the surface of the cystine functionalised AuNPs.

The explanation why cystine coating is more successful compared to cysteine coating may be due to one or several of the following reasons; I, upon adsorption of cystine, since the internal S—S bridge has to be broken initially, the formation of thiol linkages might be less randomised sterically compared to binding of cysteine. This might be unfavourable for the formation of bonds between AuNPs and the α-amines. II, in the solution, at intermediate pH the carboxylates of the cystine might be protonized to a lesser extent than the carboxylate of the cysteine and hence less cross-linking occurs. III, once bound to the surface, the cysteine shell obtained from cystine solution might acquire a different organisation compared to cysteine adsorbed from cysteine solution. This might give AuNPs with different net charge at a given pH, and hence the solutions will acquire different stability.

### Electrode Functionalization

The double tunnel junction structure of the SET sensing device can be achieved by gold electrodes covered by a thin insulating layer and the metal nanoparticle positioned there between. The best way to position the metal nanoparticle is to covalently bind it to the electrodes in a self-assembly process. By using gold electrodes covered with a thin insulating layer formed by absorption of a SAM of thiols or disulfides this is possible. Which kind of SAM to use is practically determined by the surface. As thiols and disulfides can be found with various different terminal groups, one can design the electrodes with layers that bind bare gold particles as well as functionalized ones.

As discussed above one way to functionalize surfaces with especially well defined properties are to modify them with so-called self-assembled monolayers, SAMs. The underlying reason for the formation of a SAM is partly a direct strong interaction between the molecular species and the solid support, but interactions between the molecules and the solvent or other molecules in the solution are also important. The best-understood and most well-characterised SAM-methods are those prepared from silanes on silicon or glass surfaces and those prepared from organosulfuric compounds on noble metals, predominately gold.

More complex methods for AuNP immobilisation onto gold surfaces utilizes both SAM modified surfaces and functionalised nanoparticles. For example, gold nanorods functionalised with positive charge can be assembled to a negatively charged SAM of 16-mercaptohexadecanoic acids through electrostatic interactions and ketone decorated gold nanoparticles can bind covalently to SAMs presenting aminooxy groups. A further example where biologic interaction can be utilized is the assembly of single-stranded DNA functionalised nanoparticles to surfaces modified with complementary single-stranded DNA.

### Example 4

### Preparation of a Functional Surface for the Immobilization of Functionalized AuNPs

As a surface for the immobilisation of the cystine functionalised AuNPs in Example 3, a SAM of mercaptohexadecanoic acid (HSC16OOH) was prepared on a plain Biacore gold surface. Optimally the resulting SAM should be dense, approximately 2 nm thick and display negatively charged carboxylate groups at its surface. All glassware and tweezers used were washed in solution of 5:1:1 parts of water, hydrogen peroxide (30% v/v) and NH₄OH (25% v/v) at 80° C. for at least 10 minutes followed by extensive rinsing with water.

Mercaptohexadecanoic acid (SigmaAldrich, 90%) was solved in ethanol (pure, 99.5%) to a concentration of approximately 0.2 mM. A plain gold surface (SIA Kit Au, Biacore) was cleaned in solution of 5:1:1 parts of water, hydrogen peroxide (30% v/v) and NH₄OH (25% v/v) at 80° C. for 5 minutes, whereupon it was rinsed thoroughly with water and immersed in the thiol solution over the night or longer. After incubation, the surface was rinsed with ethanol and sonicated 2-3 minutes in order to remove loosely adhered thiols, whereupon the surface was washed repeatedly and stored in ethanol.

This SAM was chosen since it forms a well-defined and isolating layer, which is desirable for the tunnelling barriers of the SET sensor. Exchanging the chosen thiol for another having a shorter carbon chain, can vary the thickness of the SAM, however this will also affect the ordering and isolating ability of the SAM. The SAM prepared displays carboxylate terminated surfaces, allowing it to be activated for carbodiimide coupling.

In order to perform a successful SET-assay, also other carboxylate terminated alkanethiols can be employed for the assembly of gold nanoparticles. However in order to ensure tunnelling, carboxylate terminated alkanethiols with 16 or less carbon atoms should be used, more preferably carboxylate terminated alkanethiols with 14 or less carbon atoms, more preferably carboxylate terminated alkanethiols with 12 or less carbon atoms, more preferably carboxylate terminated alkanethiols with 10 or less carbon atoms, more preferably carboxylate terminated alkanethiols with 8 or less carbon atoms, more preferably carboxylate terminated alkanethiols with 6 or less carbon atoms, more preferably carboxylate terminated alkanethiols with 4 or less carbon atoms.

### Covalent Immobilisation

Unlike DNA, antibodies or other proteins cannot be synthesised on the surface of a chip, but have to be immobilised onto the surface. The preparation procedure of such a miniaturised device would involve the immobilisation of peptides, proteins, antibodies or other affinity ligands onto transducer surfaces through appropriate chemical or physical treatment. Though, in contrast to nucleic acids, proteins display a much higher level of chemical and structural complexity and often react unpredictably to different immobilisation and detection strategies. Proteins or peptides can be immobilized to the active surfaces through physical absorption, electrostatic binding, covalent coupling or through a coupling protein or linker. However, physical adsorption or covalent attachment of antibodies or other proteins onto solid surfaces increase the possibility of denaturation and conformational changes. This holds especially for the case of hydrophobic physical adsorption, which is thought to induce partial or complete denaturation of most proteins. For an antibody this means total or partial loss of functionality, which leads to less sensitive and unstable sensor surfaces. Physisorbed proteins also tend to leave the surface due to gradual elution during the analytical performances causing low reproducibility of such sensors.

In order to covalently attach a peptide or a protein, the special reactive groups in the side chains of some amino acids can be employed. This includes lysine as well as the N-terminal of the peptide wearing primary amines; aspartate, glutamate and the C-terminal wearing carboxylate groups and cysteine residues having a sulfhydryl group.

The amino acids carrying carboxylate groups or amines are quite frequently occurring in most peptides and proteins including antibodies. The use of chemistry forming peptide-like linkages to these residues is hence usually an efficient and easy approach for immobilisation. On the other hand, due to the abundance of such groups, the proteins immobilised may obtain a randomised orientation. For example, some immobilised antibodies may loose antigen activity since binding limits the space needed for antigen interaction at the hyper variable regions. Even if not sterically hindered, unfavourable binding may reduce the degrees of freedom for the antibody. This indeed can decrease the antigen binding efficiency.

To overcome this problem, binding of the proteins through unique specific amino acid residues or specific groups rather than random amine groups can be a strategy. For example the sulfhydryl groups in the rarely occurring cysteine residues can be utilized. However, since they are all involved in forming disulfide bridges with one another, these bindings first have to be broken either by enzymatic cleavage of the antibody and/or by the use of mild reducing agents. In the case of synthetic, engineered peptides a cysteine residue can be placed at an appropriate position to ensure orientated immobilisation. Two alternative binding strategies can be employed for sulfhydryl groups; cysteine can be bound either through reversible disulfide bounds or through irreversible thioether bonds.

### Glutaraldehyde Coupling

One often used strategy for immobilisation of proteins is the activation of an amine containing surface with glutaraldehyde (GA). GA is a bi-functional di-aldehyde often used as a cross-linker for tissues, and it reacts with the amines of a matrix by reductive amination to form alkylated groups with terminal aldehyde (formyl) groups, see Scheme I. This group can in turn react with other primary amines, for example those from a protein. The possible points of connection are then limited to the N-terminal residue of the peptide chain(s) or to side chains of lysine residues.

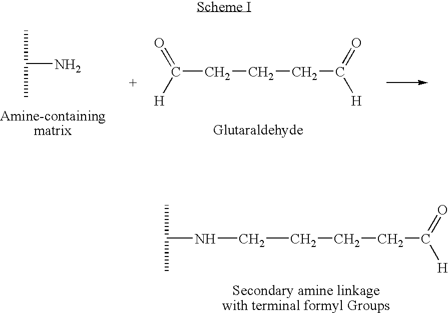

In a SET sensing device the electrodes can be covered with a layer of thiols terminated with an amino group e.g. 2-mercapto ethanol amine, which subsequently is bound to glutaraldehyde. The glutaraldehyde-modified surface will then bind covalently to gold particles functionalized with cysteine.

### Carbodiimide Coupling

Another approach for chemical immobilisation is carbodiimide coupling. Carbodiimides are special molecules, which have proven useful for the formation of peptide linkages between carboxylates and amines. For example, the N-substituted carbodiimide [1-ethyl-3-(3-dimethylaminopropyl) carbodiimide] (EDC), can react with a carboxylate group to form a highly reactive and short-lived ester intermediate. This can further react with primary amines to form amide bonds, with sulfhydryl groups to form thioesters or with water to hydrolyse back to carboxylate. In order to avoid quick deactivation, i.e. hydrolysis, N-hydroxy-succinimide (NHS) can be added. NHS provides a more stable intermediate ester increasing the coupling yield obtained, see Scheme II. Another feature of EDC/NHS compared with most other methods utilizing amine coupling, for example GA, is that it does not introduce any new linkage molecule remaining in the system.

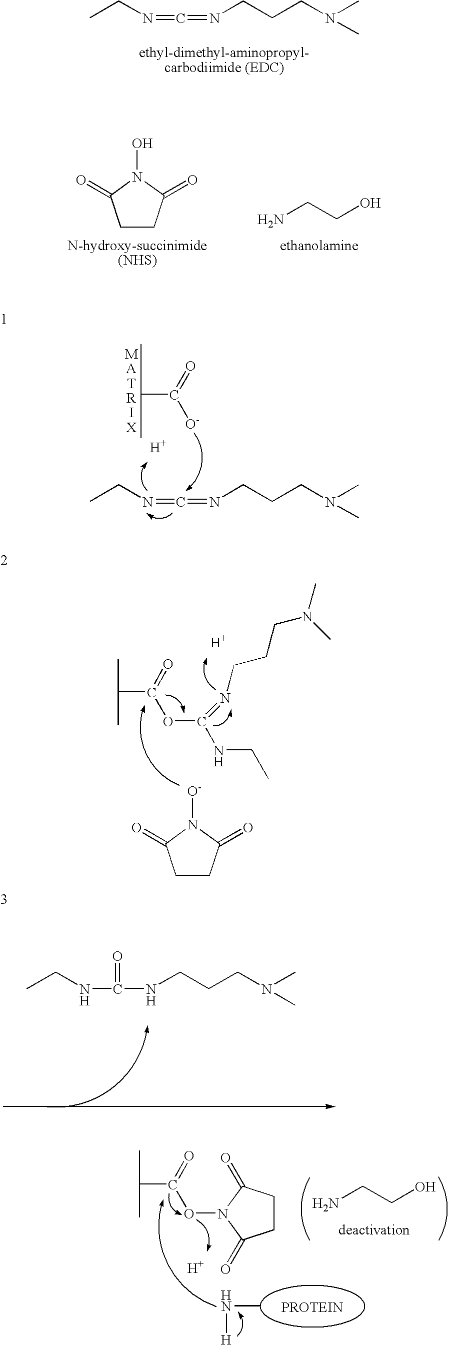

The easiest approach for immobilisation of for example a protein or other molecular species comprising a carboxylate group, using EDC/NHS-coupling is to activate a carboxylate group of a functional matrix and to bind those to the primary amines of the protein. This limits the coupling possibilities to the N-terminal residue and the lysine residues of the protein. One can also activate the carboxylate groups of the protein, i.e. those of the C-terminal, aspartate and glutamate residues and to react those with amines of a functionalised matrix, for example 3-aminopropyltriethoxysilane (APTES). However, activation with EDC/NHS in solutions containing ligands with both amines and carboxylates makes the ligands precipitate. This is especially valid for proteins, which precipitate severely in the presence of EDC and NHS. This can be overcome by activating only with EDC.

By employing the strategies discussed above i.e covalent coupling of one species, for example AuNPs functionalized with amine groups, using glutaraldehyde to specifically reactive groups on a second species having a primary amine, for example an antibody, such complex structures as nucleotides, DNA-fragments, DNA-sequences, amino acids, peptides, proteins, antibodies, enzymes, receptors and or molecular imprinted polymers can be immobilized onto surfaces which do not normally bind these structures.

### Example 5

### Immobilization of Functionalized Gold Nanoparticles and Subsequent Protein Immobilization

EDC/NHS were chosen to activate the carboxylates of the SAM prepared in Example 4 above, whereupon the cystine modified AuNPs, prepared in Example 3, can be covalently immobilised by the formation of peptide linkages between the activated carboxylates and the free amines on the surface of the cystine functionalised gold nanoparticles. In a second step, the carboxylate groups of cystine modified AuNPs, and of course also remaining SAM carboxylates, are activated with EDC/NHS, whereupon Avidin can be covalently immobilised to the surface of the already bound AuNPs. Finally, biotin-BSA is allowed to react with the immobilised Avidin in order to test the biotin binding ability for the immobilised Avidin.

A Biacore 1000 system was utilized to monitor the process of carbodiimide mediated covalent immobilisation of cystine modified AuNPs to a carboxylate terminated SAM, and the subsequent immobilisation of Avidin and biotin-BSA to the AuNP/SAM surface. A gold surface with a SAM of mercaptohexadecanoic acid as described above was rinsed with water and mounted onto a surface holder according to the manufacturers' description. The Biacore system was run using HBS running buffer (Biacore AB) and all analytes were degassed before injection. The EDC/NHS solution was always freshly prepared just before injection. The flow rate was fixed to 10 μl/min. The general procedure for immobilisation of AuNPs and proteins is described in table 1 below.

TABLE 1
Method for cys-AuNP immobilisation with Biacore

| Injection | | Injection | |
|---|---|---|---|
| order | Analyte | time | Purpose |
| 1 | EDC/NHS | 10 min | Activation of SAM |
| | (200 mM/50 mM) | | carboxylates |
| 2 | Cys-AuNP | 10 min | Covalent immobilisation |
| | (different conc.) | | of AuNPs |
| 3 | Ethanolamine | 5 min/10 min | Deactivation of |
| | (1 M) | | unreacted carboxylates |
| 4 | EDC/NHS | 10 min | Activation of |
| | (200 mM/50 mM) | | Cys-AuNP carboxylates |
| 5 | Avidin | 10 min | Immobilisation of |
| | (1 mg/ml, PBS) | | Avidin to AuNPs |
| 6 | Ethanolamine | 5 min/10 min | Deactivation of |
| | (1 M) | | unreacted carboxylates |
| 7 | Biotin-BSA | 5 min | Test functionality |
| | (1 mg/ml, PBS) | | of Avidin |

In order to test whether the Avidin binds to the cystine functionalised gold nanoparticles, it was examined how the amount of bound Avidin depended on AuNP surface coverage on the SAM. Assuming that a dynamic equilibrium exists between immobilised AuNPs and AuNPs in the reaction solution, the equilibrium surface coverage depends on the particle concentration in the solution. Since the immobilisation occurs in flowing conditions, the concentration of the particle solution can be considered constant over time. Hence, in order to achieve different AuNP surface coverage, the concentration of the particle solution was stepwise lowered by dilution with water until a change in the saturation level was observed for the AuNP signal.

To see if the presence of gold nanoparticles had significant influence on the amount of Avidin bound, the amount of Avidin immobilised directly to SAMs was compared with the amount immobilised to AuNP-functionalised SAMs displaying maximal AuNP signal, utilizing the one-tailed student's T-test. In order to characterise the procedure, different reference tests were performed where the process conditions were altered (see Reference test section below).

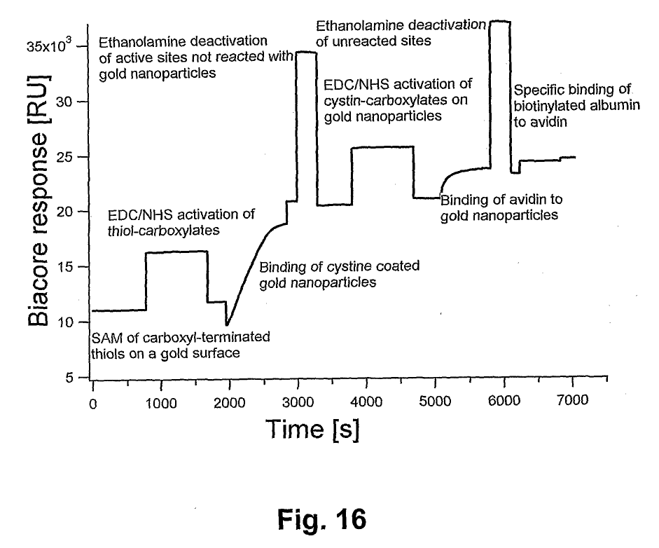

FIG. 16 shows the principle procedure for the AuNP and Avidin immobilisation as detected with the Biacore system. The activation of the carboxylate SAM usually rendered a Biacore response of approximately 600 RU. This is a little bit more than what is achieved for EDC/NHS activation of a Biacore CM5-chip (gold surface with a carboxylate modified dextran matrix extending some hundred nanometres from the surface) and is probably an effect of the large carboxylate density at the SAM surface as well as the fact that the activation takes place close to the gold surface.

The binding of cystine modified AuNPs resulted in a sigmoid like Biacore response, curve giving a maximum response around 6300 RU for injected solutions of cys-AuNPs, with concentrations spanning over a wide range. Deactivation with ethanolamine subsequent to the particle immobilisation decreased the response 100-200 RU, however larger decreases could be seen occasionally, maybe correlated with the age of the particle solution.

The second EDC/NHS activation gave about the same response as the first activation step and the curve obtained from the following Avidin immobilisation resembled what is usually obtained for EDC/NHS mediated coupling of a protein. The maximum amount of Avidin that could be immobilised to the SAM/AuNP surface corresponded to about 2700 RU, which decreased to about 1900-2000 RU after deactivation with ethanolamine. The large decrease due to the deactivation probably indicates that some of the Avidin initially had bound due to electrostatic interaction between the positively charged Avidin and the negatively charged SAM. Finally, biotin-BSA corresponding to a little bit more than 1000 RU bound quickly to the immobilised Avidin. Due to the very strong interaction between Avidin and biotin, the curve obtained from binding of biotin-BSA resembles rather a buffer shift than a protein immobilisation curve.

In order to establish that the Avidin was immobilised to the AuNPs and not only to parts of the SAM not covered with AuNPs, it was examined how the amount of immobilised Avidin depended on the amount of previously immobilised AuNPs. If the amounts of Avidin increases on the surface as the immobilised AuNPs become more abundant, it is most probably an indication that Avidin binds to the particle surfaces, since the particle surfaces provide a larger area for immobilisation compared to the flat surface. Additionally, the curvature of the particle surface may also facilitate the protein immobilisation due to sterical reasons.

Assuming that a dynamic equilibrium exists between immobilised AuNPs and AuNPs in the reaction solution, the equilibrium surface coverage would be dependent on the particle concentration in the solution. Since the immobilisation occurs under flowing conditions, the concentration of the particle solution can be considered constant over time. Therefore, in order to achieve different coverage of immobilised AuNPs on the SAM, the concentration of the gold nanoparticle solution was stepwise lowered by dilution with water before injection. For much diluted solutions, i.e. the AuNP solution appeared almost uncoloured; a decrease in the saturation level was observed for the AuNP immobilisation.

It is probable that a lower Biacore response actually corresponds to a situation with fewer immobilised AuNPs. This was also confirmed by a visual inspection of a Biacore surface for which different low concentrated solutions of cystine functionalised AuNPs were injected in the different channels. The intensities of the red bands observed decreased in correspondence with the saturation levels obtained for the different channels. FIG. 25 shows the injection of three differently concentrated solutions of cystine modified AuNPs giving raise to different AuNP surface coverage and the subsequent immobilisation of Avidin to these surfaces. From this, it is clear that surfaces with more AuNPs seem to bind more Avidin than surfaces with little immobilised AuNPs. There is a clear trend towards more Avidin immobilised and biotin-BSA bound for larger relative AuNP surface coverage, however both curves seem to reach some kind of saturation for higher values. The reason for this might be sterical hindrance as well as electrostatic repulsion between the positively charged Avidin, as the protein density grows larger on the surface.

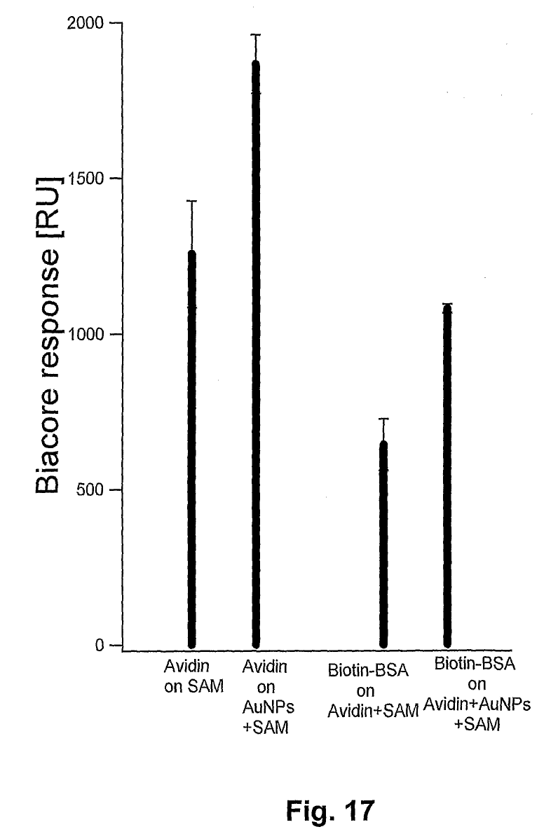

To see if the presence of gold nanoparticles had significant influence on the amount of Avidin bound, a one-tailed student's T-test was performed to compare the amount of Avidin immobilised directly to the SAM, with the amount immobilised to the SAM with maximum AuNP surface coverage. It was found that more Avidin bound to the AuNP modified SAM than what was bound to the unmodified SAM, with p<0.001, i.e. there exists a significant difference. FIG. 17 compares the mean values (n=3 and n=4 respectively) with 95% confidence intervals for the different amounts of Avidin and subsequently biotin-BSA obtained for the different surfaces. Since both Avidin and biotin-BSA have a molecular weight of approximately 66 kDa, the Biacore response at the x-axis can be exchanged for surface density. Table 2 summarizes some important features obtained from the data in FIG. 27.

TABLE 2
Avidin and biotin-BSA on the SAM and SAM/AuNP surface

| | Avidin | Biotin-BSA | Avidin surface | Biotin- |
|---|---|---|---|---|
| | (mean ± 95% | (mean ± 95% | coverage | BSA/ |
| | Cl) pg/mm² | Cl) pg/mm² | (d = 5 nm) % | Avidin |
| SAM | 1255 ± 171 | 642 ± 82 | 23.2 | 0.51 |
| SAM + | 1864 ± 94 | 1078 ± 14 | 34.5 | 0.58 |
| AuNPs | | | | |
| Change | +48.5% | +67.8% | +48.5% | +13.0% |

The amount of immobilised Avidin (mean value) increases about 48.5% percent for the AuNP modified SAM compared to the SAM only. Regarding the amount of biotin-BSA that bound to the immobilised Avidin, the increase is even larger, about 67.8%. This represents an increase of the biotin-BSA to Avidin ratio from 0.51 to 0.58, i.e. Avidin bound to the AuNP modified SAM seems to bind more biotin-BSA than Avidin bound directly to the SAM. This may reflect a more favourable sterical configuration of AuNP bound Avidin compared to Avidin bound to the SAM, allowing more biotin-BSA to interact with each Avidin.

Approximating the diameter of each Avidin to 5 nm, gives a relative surface coverage of 23.2% for Avidin immobilised to the SAM, which increases to 34.5% for Avidin immobilised to the AuNP modified SAM. This does not represent a full monolayer, however a significant part of it. The surface coverage of Avidin is obviously dependent on the number and density of activated carboxylate groups obtained in the activation step. However, electrostatic repulsion between the positively charged Avidins may also contribute to a lower surface coverage. One way to overcome this would be to alter the pH, however the isoelectric point for Avidin is as high as 10.5, which is too high for carbodiimide coupling chemistry.

As can be seen from the discussion above there are many ways to attain chemical or biological function of the SET-sensor. This is due to the versatility in gold nanoparticle functionalization. By the functionalization of the nanoparticle the possibilities for immobilizing molecules for use in detection of chemical reactions increase enormously. The functionalization provides the gold nanoparticles with chemical handles which enable binding of almost any molecular structure, whether it is through physical adsorption, electrostatic binding, covalent coupling or through a coupling protein or linker.

### Reference Tests

In order to see whether the cystine modified AuNPs interacted directly with the carboxylate terminated SAM, cystine functionalised AuNPs were injected directly on the SAM. The Biacore response did not show anything at all besides the buffer shift, i.e. no interaction with the surface could be seen during the injection period and no detectable change was seen for the baseline level after the injection. This indicates that the cystine coated AuNPs indeed are stabilised through a net negative charge, which also prevents them from approaching and interacting with the negatively charged SAM.

Since Avidin at normal pH is positively charged whereas the carboxylate terminated SAM and the cystine coated AuNPs are negatively charged, it is likely that electrostatic interaction between the surface/AuNPs and the Avidin would occur. In order to test for electrostatic interaction between Avidin and the carboxylate terminated SAM or the SAM with immobilised cystine modified AuNPs, Avidin solution was injected directly on the SAM and the SAM/AuNP surfaces respectively without previous EDC/NHS activation. The Avidin injections were followed by injections of ethanolamine (1.0 M) and/or KCl₂ (1.0 M) in order to “salt out” electrostatically bound proteins.

To clarify that the EDC/NHS activation actually led to covalent immobilisation, i.e. that the Avidin binding not solely was due to electrostatic interactions, in addition to ethanolamine also KCl₂ (1 M) was injected after EDC/NHS immobilisation. The general features obtained from the Biacore response curves are summarized in table 4. It was shown that indeed a lot of Avidin bound to the SAM, as well as the AuNP modified SAM, in the absence of activation. However, this binding clearly differed from the binding via EDC/NHS regarding the fact that it went on quickly, yielding a lot of bound Avidin, which however could be salted out by ethanolamine or KCl₂. The EDC/NHS mediated binding was slow and yielded relatively low amounts of bound Avidin that only came loose to a minor extent by addition of ethanolamine or KCl₂. Hence, it can be concluded that electrostatic binding is not responsible for any major part of the Avidin binding when EDC/NHS is used.

TABLE 4
Reference tests

| | Response after | Response after | |
|---|---|---|---|
| | Avidin | Ethanolamine/KCl₂ | Rate of |
| Surface | (RU) | (RU) | reaction |
| SAM | 2525 | 330 | Fast |
| SAM + EDC/NHS | 1564 | 1200 | Slow |
| SAM + AuNPs | 3246 | 625 | Fast |
| SAM + AuNPs + | 1850 | 1470 | Slow |
| EDC/NHS | | | |

### Immobilisation of Functionalized Gold Particles to Nanosized Electrodes

The preparation of sensor chips for the SET-assay requires that the AuNPs can be immobilised at the gap between the nanofabricated sensor electrodes. Hence, the protocol used above for AuNP immobilisation with the Biacore system had to be adapted in order to allow bench-top immobilisation of the cystine functionalised AuNPs to the small, lithographically defined gold surfaces of the sensor chips. The carboxylate terminated SAM was formed directly on the gold areas of the sensor electrodes by immersing the sensor chips in the thiol solution. At the sensor interface, the SAM is not only supposed to function as foundation for AuNP coupling, but also to function as tunnelling barrier, electric isolator against electrolytes, and to prevent spontaneous migration of gold from the electrodes.

The used thiol, mercaptohexadecanoic acid, was not chosen with respect to all these factors, why it is not certain that this is the most appropriate in order to perform a SET-assay. Nevertheless, for comparison the mercaptohexadecanoic acid was used also for the bench-top immobilisation. The nanosized electrodes of the sensor chips were too delicate to undergo any tough washing procedure; however, the chips were not exposed to air otherwise than in the clean room after evaporation, whereupon the chips were kept in ethanol until the AuNP immobilisation. All glassware and tweezers were washed in solution of 5:1:1 parts of water, hydrogen peroxide (30% v/v) and NH₄OH (25% v/v) at 80° C. for at least 10 minutes followed by extensive rinsing with water before use.

Sensor chips were immersed in mercaptohexadecanoic acid (0.2 mM in ethanol 99.5%) over night or longer, whereupon the chips were extensively rinsed with pure ethanol and water. Chips were activated in freshly prepared solution of EDC and NHS (200 mM and 50 mM in water respectively) for 25 minutes, whereupon the chips were rinsed with water and transferred to concentrated solution (hardly transparent) of cystine functionalised AuNPs for 30 minutes. After particle immobilisation, the chips were carefully washed with water during 30 minutes in order to remove loosely adhered particles. The chips were stored in water until use, then the chips were dried under N₂ flow. The chips were subject to morphological examination with SEM and electrical measurements.

These particular electrodes were only briefly washed with water after the immobilisation, and what can be observed are conjugates of AuNPs and thiols loosely adhered to the surface. Most of the electrode structures are not made of gold; the gold is restricted to the small area closest to the gap and consequently most of the AuNP-thiol aggregates are assembled there. By immersing the electrodes in water (gentle stirring) for about 30 minutes, most of the seen AuNPs come loose, whereupon the remaining particles hardly can be seen using the SEM.

Electrodes with immobilised cystine functionalised AuNPs were continuously subject to electrical measurements in order to perform a SET-assay. However, in order to enhance tunnelling the preparation with mercaptohexadecaonoic acid was exchanged for preparation with mercaptopropionic acid. In order to increase the yield of functional SET-structures, the number of particles immobilised to the surface was increased by performing the activation and binding step as described above two or more times repeatedly.

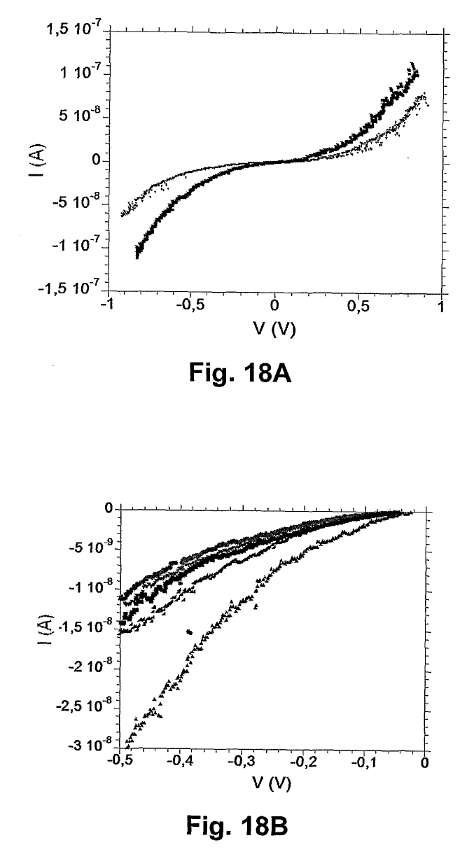

Nano fabricated electrodes were contacted in a specially designed system and the IV-characteristics were measured. Immobilisation of cystine modified AuNPs to the electrodes gave rise to Coulomb blockade by measurement in distilled water. After addition of Avidin to the particle immobilised to the electrodes, a change of the IV-characteristics could be detected, see FIG. 18A. Monitoring this shift as a function of time after addition of the protein, see FIG. 18B, revealed a continuous shift in the IV-characteristics, mirroring the slow adsorption of the macromolecules to the surface. The shift eventually reaches a saturation level where no more Avidin adsorbs to the surface. The existence of a course of adsorption is different from what can be seen when for example exchanging the fluid (e.g. exchanging water for ethanol) in the SET-assay giving rise to an immediate shift of the Coulomb blockade due to the fluids' different dielectric properties (results not shown). Therefore, the slow course of the shift is a clear indication that the obtained shift is due to macromolecular adsorption and it also demonstrates the feasibility of the SET technique for measurements on biomolecules.

The feasibility of the device for biosensing applications is further demonstrated in FIG. 6. FIG. 6 shows the IV-characteristics obtained for electrodes with immobilised cystine modified AuNPs in buffer solution before (A) and after addition of Avidin to the system (B). After rinsing with buffer, biotinylated albumin was added to the surface giving rise to a further change of the coulomb blockade (C). By exchanging the biotinylated albumin for any other biotinylated protein or peptide, e.g. an antibody, Avidin may be used as a general platform for coupling of biological active agents to the active site of the sensor.

### Alternative Embodiments of Mechanical Design

It should be understood by the person skilled in the art that the mechanical design of the electrodes can be made in alternative forms as long as the physical dimensions between electrodes allow for insertion of at least one activation object 4 and allow for tunneling current to pass through the activation object 4. In the present invention the gap 12 between electrodes are of the order a few nanometers; however, the optimal distance is of course depending on the voltage applied between the electrodes and the type of activation object 4.

### Applications of the Present Invention

The present invention may find use in a vast number of different applications since it is possible to detect very small amounts of molecules or particles. These applications span from DNA sequence determination or detection of single DNA parts, blood sample analysis, protein analysis, pollution detection in air or water, exhaust purification as part of the cleaning process or as a quality assurance, detection of allergens or toxic substances for instance in food industry or during industrial processes, for security purposes, and drug detection for stopping illegal import of drugs. The above mentioned application areas are only meant as examples and the person skilled in the art may find many more areas of interest where the present invention may find applicability.

### Some Concluding Remarks

For the case of using thiol chemistry for the active site it is optimal to use gold electrodes, see FIG. 3 below. When the surface is an oxidized silicon wafer a sticking layer is needed in order for the gold to adhere to the surface. Such sticking layer could be for example chromium, titanium, NiCr, or titanium oxide. Since the thiols do not bind to the most commonly used sticking layer in a satisfying way, it is important that the gold film fully covers the sticking layer in the narrow gap of the active site. One way to make sure it does is to use angle evaporation, an example of this is shown in FIG. 2 above.

In order to avoid the sticking layer a layer of aluminum oxide could be evaporated on top of the surface. By doing so, the risk of gold not covering the sticking layer is eliminated. Gold adheres to aluminum oxide and no sticking layer is needed.

An alternative embodiment of the present invention may be a structure wherein a gate electrode 930 connected to a voltage source is located close to a tunnelling particle 904 and where the gate is arranged to receive particles to be sensed and the presence of these particles change the electrical field around the tunnelling object and thus the tunnelling current through the tunnelling object will vary depending on the concentration of particles on the gate 930. Moreover another electrode may be used to direct the electrical field from the gate.

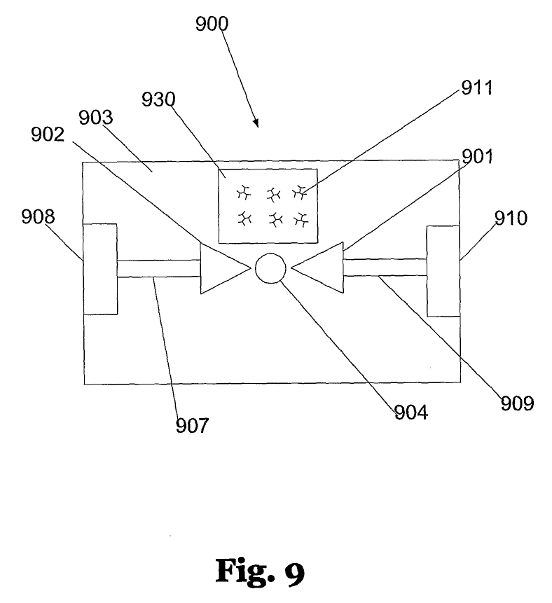

The described microelectronic (or nano electronic) device 900 is shown schematically in a top view in FIG. 9. Two electrodes 901 and 902 are located with a gap between them and a tunnelling object 904 is located in this gap. Each electrode is connected via a conducting line 907, 909 to a respective contact pad 908, 910. The gate 930 is arranged with suitable receiving objects 911 that specifically may receive a particular particle/substance of interest to detect the presence of. Otherwise this embodiment will operate in the same manner as described above for the embodiment schematically illustrated for FIG. 1b except for the fact that the potential of the gate 930 can be regulated.

With the term particle is meant a unit of a substance, a molecule, an atom, or similar object. For instance, complex molecules like DNA and proteins may be detected, or complex molecules like explosives may also be detected. In some configurations it is possible to detect single atoms with a suitable receptor.

With the term “microelectronic device” is meant a device fabricated with similar fabrication methods as used for MEMS/NEMS devices, i.e. small scale integrated electrically connectable sensing devices.

The activation object 4 is in all above examples at least partly made of gold; however other materials may be used for instance titanium, aluminium, copper, iron, silver, palladium, cobalt, cadmium selenide, or composition of materials. However, the invention is not limited to the above exemplified materials other may be used depending on sought functionality of the sensing device 20.

It should be noted that the word “comprising” does not exclude the presence of other elements or steps than those listed and the words “a” or “an” preceding an element do not exclude the presence of a plurality of such elements. It should further be noted that any reference signs do not limit the scope of the claims, that the invention may be implemented in part by means of both hardware and software, and that several “means”, “devices”, and “units” may be represented by the same item of hardware.

The above mentioned and described embodiments are only given as examples and should not be limiting to the present invention. Other solutions, uses, objectives, and functions within the scope of the invention as claimed in the below described patent claims should be apparent for the person skilled in the art.

1. An electronic sensing device for sensing at least one particle (13), comprising at least two electrodes (1, 2) positioned with a gap (12) formed between said electrodes (1, 2) and an activation object (4) positioned in said gap with an insulating layer between said activation object (4) and each electrode (1, 2); said activation object being able to transfer electrons and arranged with at least one binding structure (11) bonded to said activation object (4) for receiving said at least one particle (13) characterized in that said electrodes are formed with an inter distance of less than 50 nm and said electrodes (1, 2) being connectable (7, 8, 9, 10) directly or indirectly to a signal acquisition system (203); said sensing device is arranged to allow a tunnelling current related to the presence of said at least one particle (13) in said binding structure (11), to flow through said activation object (4).

2. The device according to claim 1, further comprising an insulating layer (5, 6) formed on at least part of at least one electrode (1, 2) on a surface of said electrode (1, 2) facing particles to be sensed.

3. The device according to claim 2, wherein said insulating layer (5, 6) is formed in part by angle evaporation on a double resist mask.

4. The device according to claim 3, wherein said insulating layer (5, 6) is made of SiO₂, titanium oxide, aluminium oxide, chromium oxide, iron oxide, beryllium oxide, ceramics, polystyrene or Teflon.

5. The device according to claim 1, further comprising a sticking layer (14) formed under at least part of each electrode (1, 2).

6. The device according to claim 1, wherein said sticking layer is made of at least one of chromium, titanium, NiCr, or aluminium oxide.

7. The device according to claim 1, wherein said activation object (4) is a nano sized particle made of a metal or a conducting compound.

8. The device according to claim 7, wherein said activation object (4) is made of at least one of gold, titanium, aluminium, copper, iron, silver, palladium, cobalt or cadmium selenide.

9. The device according to claim 1, wherein said activation object (4) is stabilized by a stabilizing agent.

10. The device according to claim 9, wherein said stabilizing agent is citrate.

11. The device according to claim 1, wherein said activation object (4) is stabilized and/or functionalized with a self-assembling monolayer (SAM).

12. The device according to claim 11 wherein the self-assembled monolayer, SAM. comprises one or more thiols.

13. The device according to claim 12, wherein the self-assembled monolayer, SAM comprises one or more alkanethiols.

14. The device according to claim 13, wherein the self-assembled monolayer, SAM is assembled from hydrophilic substituted alkanethiols or hydrophobic alkanethiols.

15. The device according to claim 9, wherein said stabilized activation object (4) has a diameter of less than 20 nm, more preferably a diameter of less than 18 nm, more preferably a diameter of less than 16 nm, more preferably a diameter of less than 14 nm, more preferably a diameter of less than 12 nm, more preferably a diameter of less than 10 nm, more preferably a diameter of less than 8 nm, more preferably a diameter of less than 6 nm, and most preferably a diameter of less than 4 nm.

16. The device according to claim 1, wherein said activation object (4) is functionalized by binding a binding structure (11).

17. The device according to claim 10, wherein the stabilized activation object in claim 10 has been functionalized by exchange mediated functionalisation.

18. The device according to claim 16, wherein binding structure (11) is a compound from the group comprising water solvable ionic or zwitterionic compounds.

19. The device according claim 16, wherein the binding structure (11) is a molecular structure having functional groups chosen from the group comprising thiol, sulphide, amine, carboxylate, cyanide, diphenylphosphine and/or pyridine functional groups.

20. The device according to claim 16, wherein the binding structure (11) is chosen from the group comprising ions, atoms, molecules, low-molecular compounds, nucleotides, DNA-fragments, DNA-sequences, amino acids, peptides, proteins, antibodies, enzymes, receptors, and/or molecular imprinted polymers.

21. The device according to claim 16, wherein the activation object (4) has been functionalized with Avidin.

22. The device according to claim 21, wherein the avidin functionalized activation object (4) is bound to a biotinylated protein or protein fragment.

23. The device according to claim 16, wherein the activation object (4) has been functionalized with cysteine.

24. The device according to claim 16, wherein the activation object (4) has been functionalized with cystine.

25. The device according to claim 1, wherein the surfaces of said electrodes (1, 2) have been functionalized.

26. The device according to claim 25, wherein said functionalized electrodes (1, 2) are covered with a self-assembled monolayer, SAM.

27. The device according to claim 26, wherein said self-assembled monolayer, SAM comprises one or more alkanethiols with 16 or less carbon atoms, preferably alkanethiols with 14 or less carbon atoms, preferably alkanethiols with 12 or less carbon atoms, preferably alkanethiols with 10 or less carbon atoms, preferably alkanethiols with 8 or less carbon atoms, preferably alkanethiols with 6 or less carbon atoms, preferably alkanethiols with 4 or less carbon atoms.

28. The device according to claim 27, wherein said alkanethiol is a substituted alkanethiol.

29. The device according to claim 27, wherein said alkanethiol is a carboxylate terminated alkanethiol.

30. The device according to claim 29, wherein said alkanethiol is mercap-tohexadecanoic acid

31. The device according to claim 29, wherein said alkanethiol is mercaptopropionic acid.

32. The device according to claim 16, wherein the activation object (4) is a functionalized activation object (4) immobilized to an electrode (1, 2) whose surface has been functionalized.

33. The device according to claim 32, wherein said functionalized activation object (4) is immobilized to a functionalized electrode by covalent immobilization.

34. The device according to claim 32, wherein said functionalized activation object (4) is immobilized to a functionalized electrode by carbodiimide coupling.

35. The device according to claim 32, wherein said functionalized activation object (4) is immobilized to a functionalized electrode by glutaraldehyde coupling.

36. The device according to claim 32 wherein said functionalized activation object (4) is covalently coupled to a binding structure (11).

37. The device according to claim 36, wherein said binding structure (11) is one of the group comprising nucleotides, DNA-fragments, DNA-sequences, amino acids, peptides, proteins, antibodies, enzymes, receptors, molecular imprinted polymers.

38. The device according to claim 36, wherein said binding structure (11) is covalently coupled to a through the reactive groups of amino acid chosen from the groups comprising lysine, the N-terminal of the peptide with primary amines, aspartate, glutamate, the C-terminal with carboxylate groups and/or cysteine

39. The device according to claim 36, wherein said binding structure (11) is covalently coupled by carbodiimide coupling.

40. The device according to claim 36, wherein said binding structure (11) is covalently coupled by glutaraldehyde coupling.

41. A method for producing a cystine functionalized activation object (4) characterized in that; a) mixing equal volumes of citrate stabilized gold nanoparticles having a mean diameter of less than 20 nm and a saturated cystine solution; b) incubating the mixture in room temperature for 8-12 hrs; c) centrifuging the mixture forming a pellet; and d) redissolving the pellet in water.

42. A cystine functionalized activation object (4) prepared by a) mixing equal volumes of citrate stabilized gold nanoparticles having a mean diameter of less than 20 nm and a saturated cystine solution; b) incubating the mixture in room temperature for 8-12 hrs; c) centrifuging the mixture forming a pellet; and d) redissolving the pellet in water.

43. (canceled)

44. A system for measuring low quantities of molecules or particles comprising: an electronic sensing device (201) for sensing particles (13), comprising at least two electrodes (1, 2) positioned with a gap (12) formed between said electrodes (1, 2) and an activation object (4) positioned in said gap with an insulating layer between said activation object (4) and each electrode (1, 2); said activation object being able to transfer electrons and arranged with at least one binding structure (11) bonded to said activation object (4) for receiving at least one particle (13) characterized in that said electrodes are formed with an inter distance of less than 50 nm and said electrodes (1, 2) being connectable (7, 8, 9, 10) directly or indirectly to a signal acquisition system (203); said sensing device is arranged to allow a tunnelling current related to the presence of particle or particles (13) in said binding structure (11), to flow through said activation object (4); electronics for signal processing (203); and—a processing device (202) for control of measurement and signal acquisition for processing and analysis of measured signals.

45. The system according to claim 44, further comprising a holder (210) for holding the electronic sensing device (201) and arranged with a quick release lock.

46. The system according to claim 44, further comprising a delivery system (204) for providing particles to be measured to said electronic sensing device (201).

47. A method of fabricating a gap (806) between electrodes in an electronic sensing device (20) for sensing particles, comprising the steps of:
forming a first electrode (802) onto a surface (801);
forming an aluminium layer (805) on said first electrode (802);
oxidizing said aluminium layer (805);
forming a second electrode (804) at least partly over said first electrode
(802) and said oxidized aluminium layer (803); removing a part of said second electrode located on said oxidized aluminium layer (803); and removing said oxidized aluminium layer (803) and said aluminium layer (805) from said first electrode (802).

48. An electronic sensing device (900) for sensing particles, comprising at least two electrodes (901, 902) positioned with a gap formed between said electrodes (901, 902) and a tunnelling object (904) positioned at least partly in said gap with an insulating layer between said tunnelling object (904) and each electrode (901, 902); said tunnelling object (904) being able to transfer electrons, said device (900) further comprising a gate (930) arranged to receive particles to be sensed, characterized in that said electrodes (901, 902) are formed with an inter distance of less than 50 nm and said electrodes (901, 902) being connectable (907, 908, 909, 910) directly or indirectly to a signal acquisition system (203); said sensing device is arranged to allow a tunnelling current related to the presence of particle or particles on said gate (930), to flow through said tunnelling object (904).

49. Method of fabrication of nanogaps according to a process wherein a double resist layer is used, comprising the steps of:
patterning a top resist with electrons and developing;
developing the non-electron sensitive bottom resist layer under the top resist and forming a thin bridge of the top resist; —defining, during evaporation the distance between two evaporated electrodes, the width of the resist bridge;
forming, due to migration, grains between the electrodes; and
forming a nanogap since the grains extend the electrodes and the nanogap is formed between grains.

50. The method according to claim 49, wherein said grains are modified by a plasma.

51. A method of fabricating nanogaps comprising the steps of:
evaporating a first electrode (702) onto a surface (701); —forming an oxidized aluminium layer (703) on said first electrode (702);
forming a second electrode (704) on said surface (701) and partly on said oxidized aluminium layer (703); and
removing said oxidized aluminium layer (703) forming a gap between said first and second electrodes.
